(19) United States
(12) Patent Application Publication
(10) Pub. No.: US 2003/0158560 A1
(43) Pub. Date: Aug. 21, 2003

(54) Corrective intraocular lens system, intraocular lenses, and lens handling and installation devices for use therewith, and installation method

(76) Inventor: Valdemar Portney, Tustin, CA (US)

Correspondence Address: Howard R. Lambert, 5245 Gatewood Lane, Anaheim, CA, 92807, US

(21) Appl. No.: 10/306,352

(22) Filed: Nov. 27, 2002

Related U.S. Application Data
(63) Continuation-in-part of application No. 09/522,525, filed on Mar. 10, 2000, now Pat. No. 6,537,281.
Application No. 09/522,525 is a continuation-in-part of application No. 09/273,478, filed on Mar. 22, 1999, now Pat. No. 6,197,058.

(51) Int. Cl. A61F 9/00

(52) U.S. Cl. 606/107

(57) ABSTRACT

A method is disclosed for modifying the optical characteristics of a intraocular lens implanted in a patient's eye, the primary intraocular lens having a primary optic with a narrow annular recess formed into an anterior surface generally parallel to the anterior surface and relatively adjacent the primary optic periphery, an entrance to the recess having an overhanging lip directed toward the primary optic optical axis. The method includes forming a thin, elastically deformable secondary optic having a diameter substantially equal to the diameter of the primary optic recess entrance, and having first and second tabs extending radially outward at generally opposite peripheral regions for inserting into the primary optic recess. A secondary optic manipulation hole is formed in each of the tabs and small first and second, spaced far apart ocular incisions are made in opposite regions of the patient's eye. After inserting the secondary optic into the patient's eye with the secondary optic posterior surface positioned on the primary optic anterior surface and with the first and second tabs in close proximity with corresponding ones of the first and second ocular incisions, a first installation instrument positions the secondary optic so that the first tab is adjacent a selected region of the primary optic recess, and a second instrument preferably holds the primary optic in a fixed position; the first instrument then positions the secondary optic so the first tab is inserted into the primary optic recess. The two instruments are then repositioned for inserting the second tab into the primary optic recess in the same manner. A secondary optic is disclosed as are the two installation instruments.

This application is a continuation-in-part (CIP) of application Ser. No. 09/522,525, filed Mar. 10, 2000, which is a continuation-in-part (CIP) of application Ser. No. 09/273,478, filed Mar. 22, 1999 (now U.S. Pat. No. 6,197,058, issued Mar. 6, 2001).

### BACKGROUND OF THE INVENTION

1. Field of the Invention

The present invention relates generally to the field of ophthalmics, more particularly to ophthalmic devices, and still more particularly to dual intraocular (IOL) lens systems and associated instruments for the implanting of the same.

2. Background Discussion

At the onset it may helpful to the understanding of the present invention to define the terms “phakic” and “aphakic” as relate to human eyes. The term “phakic” is applied to an eye in which the naturals ocular lens—whatever its condition—has not been removed. In contrast, the term “aphakic” is applied to an eye from which the natural ocular lens has—for any reason—been removed. In this regard, a phakic eye is considered a dynamic or active eye because the natural lens is subject to change over time. In contrast, an aphakic eye is considered a static eye because the natural lens has been removed.

Vision in an eye is enabled by the cornea and the natural lens (and/or an implanted IOL) located posterior of the cornea, both or all of which refract light from a viewed image to the retina of the eye.

One serious and relatively common vision problem is reduced or complete loss of sight due to the natural ocular lens becoming cloudy or opaque—a condition referred to as cataract. The formation of cataracts is most often associated with natural bodily aging processes—perhaps caused or aggravated by long-term exposure to ultraviolet rays from the sun. In any case, most individuals over the age of about 60 years suffer from cataracts at least to some extent.

The current state of ophthalmics, as far as is known to the present inventor, is that cataracts cannot be cured or reversed, nor can the cataract formation process be significantly arrested. Consequently, when a natural lens becomes so cloudy by cataracts (or by any other mechanism) that the lens can no longer effectively refract light from a viewed image to the retina, thereby significantly impairing vision, the corrective action involves the surgical removal of the natural lens. In this manner, a phakic eye becomes an aphakic eye.

After the defective natural lens has been surgically removed, the common current practice is to implant in the individual's aphakic eye an artificial lens called an intraocular lens or IOL. Previously, thick, high diopter spectacles were prescribed for aphakic eyes, such spectacles however being generally disliked by most patients for obvious reasons.

Intraocular lenses are constructed from biocompatible optical materials and are, to the extent possible, configured to provide the optical characteristics (with the current exception of accommodation) of the removed natural lens in its prior healthy condition.

IOLs are generally considered to have become practical as a result of the World War II discovery by Dr. Peter Ridley that shards of damaged British fighter aircraft canopies made of PERSPEX (i.e., PLEXIGLAS)—an optically clear, hard poly-methyl methacrylate (PMMA) plastic material—embedded in pilots' eyes caused no adverse reaction in the eyes.

As a result of this discovery, hard, rigid IOLs were constructed from an optical grade of PMMA. These rigid IOLs were compatible with then-current surgical procedures used for removing natural lenses in one piece. That is, the PMMA IOLs could be implanted through the relatively large, 5-6 mm, ocular incisions made for removal of the natural lenses.

Subsequently in the early 1970's Dr. Charles Kelman developed a lens-removal procedure utilizing ultrasound to break up natural lenses. This enabled the natural lenses to be extracted with an irrigating fluid in an emulsified condition from the eye through a much smaller ocular incision than that previously needed to extract the natural lens in one piece. This advantageously resulted in reduced patient trauma and patient recovery time.

This new surgical procedure, called phacoemulsification, created a need for elastically-deformable IOLs that could be rolled or folded for insertion into the eye through the same small ocular incision used in the phacoemulsification lens removal procedure, and which then unfolded to their original shape once in the eye. Such deformable IOLs are commonly constructed from an optically clear, high refractive index, biocompatible silicone or acrylic material.

In addition to the implanting of IOLs in aphakic eyes to restore vision after removal of the natural lens, there has recently been an interest in implanting IOLs in phakic eyes to correct vision deficiencies even with healthy natural lenses. The implanting of IOLs in phakic eyes is an often-attractive alternative to some individuals to the wearing of corrective spectacles or contact lenses or having such corneal surgical procedures as radial keratomy (RK) or photo-radial keratectomy (PRK) performed.

In an aphakic eyes, an IOL is now most commonly implanted in the posterior chamber of the eye in the general location from which the natural lens was removed. Nevertheless, the implanting of an IOL in the anterior chamber is sometimes necessary because, for example, of damage to the posterior wall of the capsular bag during removal of the natural lens. In contrast, an IOL for a phakic eye is most commonly implanted in the anterior chamber of the eye, but may sometimes be implanted in the posterior chamber or on top of the natural crystalline lens.

Regardless of the reason for the implanting of an IOL or the location of the implanted IOL, a principal objective of the present invention is to provide an IOL system in which corrections to IOL spherical, cylindrical and/or add power can be easily made with minimal invasive action. Another, major objective of the present invention is to provide a method utilizing a secondary optic for modifying the optical properties of a primary optic and to provide an alternative secondary optic and instruments for attaching the secondary optic to the primary optic.

### SUMMARY OF THE INVENTION

In accordance with the present invention, there is provided a method for modifying the optical characteristics of a primary intraocular lens implanted in a patient's eye, the primary intraocular lens having a primary optic with a narrow annular recess formed into an anterior surface generally parallel to the anterior surface and relatively adjacent the periphery of the primary optic, the entrance to the annular recess having an overhanging lip directed toward an optical axis of the primary optic.

The method comprises the steps of forming a thin, elastically deformable secondary optic having a diameter substantially equal to a primary optic diameter at the primary optic annular recess entrance, and having an insertion tab extending radially outward from the secondary optic peripheral edge, the tab being sized for insertion into the primary optic annular recess, and including forming a manipulation hole in the tab.

Included in the method are the steps of making small, first and second, spaced far apart ocular incisions in the patient's eye, and inserting the secondary optic into the patient's eye with the secondary optic posterior surface positioned on the primary optic anterior surface, and with the tab in close proximity to one of the first and second ocular incisions.

Further included in the method are the steps of forming a secondary-optic-to-primary-optic attachment instrument, inserting the instrument through the first incision into the patient's eye until an instrument tip thereof engages the manipulation hole in the secondary optic tab, and positioning the secondary optic by the first instrument tip until the tab is adjacent a selected region of the primary optic recess and further positioning the secondary optic by the instrument tip so the tab is inserted into the primary optic annular recess.

The method preferably includes the further steps of forming a second secondary optic-primary optic attachment insertion instrument and inserting the second instrument through the second ocular incision into the patient's eye so that an instrument tip thereof engages the primary optic peripheral edge adjacent the tab to maintain the primary optic in a fixed position while positioning the secondary optic by the first-mentioned instrument tip so the tab is inserted into the primary optic recess.

The step of forming the first-mentioned and second secondary-optic-to-primary-optic attachment instruments preferably includes forming each of the instruments having an elongate, slender insertion needle and forming a short, axially extending pin and a pair of short axially extending ears to sides of the pin at a distal end of the needle of at least the first-mentioned instrument needle.

The step of forming the first-mentioned instruments may include forming each of the instruments having an elongate, slender insertion needle and forming an elongate, axially extending pin at the distal end of the second instrument for engaging and lifting a region of the annular recess overhanging lip adjacent the tab to facilitate insertion of the tab into the recess and forming an elongate, axially extending tongue for engaging the primary optic peripheral edge to hold the primary optic in position while the tab is inserted into the annular recess.

The method may also include forming each of the first-mentioned and second instruments having an elongate slender hollow insertion needle and axially slidingly installing a flexible pin through each of the needles, each of the pins being individually extendable and retractable in the associated needle. Preferably, included is the step of forming each of the installed pins with a curved distal end region and of a shape memory material so that when each of the pins is individually retracted into the associated needle a short exposed distal end of each pin is relatively straight and so that when each of the pins is individually extended from the associated needle a longer exposed distal end region of each pin is curved.

The method preferably also includes the step of inserting the secondary optic through one of the first and second ocular incisions.

It is preferred that the step of forming a thin, elastically deformable secondary optic, includes forming an arcuate recess in the periphery of the secondary optic adjacent each side of the tab so as to provide a secondary optic guiding region for positioning of the secondary optic by one of the insertion instruments.

More particularly, there is provided a method for modifying the optical characteristics of a intraocular lens implanted in a patient's eye that comprises the steps of forming a thin, elastically deformable secondary optic having a diameter substantially equal to a primary optic diameter at the primary optic annular recess entrance, and having first and second insertion tabs extending radially outward at generally opposite peripheral edge regions, the tabs being sized for fitting into the primary optic annular recess, and forming a manipulation hole in each of the secondary optic first and second tabs. Included are the steps of making small, first and second, spaced far apart ocular incisions in opposite regions of said patient's eye, and inserting the secondary optic into the patient's eye with the secondary optic posterior surface positioned on the primary optic anterior surface, and with the first and second tabs in close proximity with corresponding ones of the first and second ocular incisions.

The method includes the further steps of forming first and second secondary-optic-to-primary-optic attachment instruments; inserting the first instrument through the first incision into the patient's eye until an instrument tip thereof engages the manipulation hole in the first tab; and positioning the secondary optic by the first instrument tip until the first tab is adjacent a selected region of the primary optic recess. Then inserting the second instrument through the second ocular incision into the patient's eye so that an instrument tip thereof engages the primary optic peripheral edge adjacent the first tab to maintain the primary optic in a fixed position and positioning the secondary optic by the first instrument so the first tab is inserted into the primary optic recess.

The step of forming the first and second instruments includes forming each of the instruments having similar instrument tips and including the further steps of repositioning the second instrument so that the instrument tip thereof engages the manipulation hole in the second tab and repositioning the first instrument so that the instrument tip thereof engages the primary optic peripheral edge adjacent the second tab for maintaining the primary optic in a fixed position. Then included is the step of pushing the second optic by the second instrument tip until the second tab is inserted into the primary optic annular recess.

Alternatively, the step of forming the first and second instruments includes forming each of the instruments having different instrument tips and including the further steps of inserting the first instrument through the second incision into the patient's eye until an instrument tip thereof engages the manipulation hole in the second tab and positioning the secondary optic by the first instrument tip until the second tab is adjacent a selected region of the primary optic annular recess. Then inserting the second instrument through the first ocular incision into the patient's eye so that an instrument tip thereof engages the primary optic peripheral edge adjacent the second tab for maintaining the primary optic in a fixed position, and positioning the secondary optic by the first instrument tip so the second tab is inserted into primary optic annular recess.

The step of inserting the second instrument through the second and first ocular incisions includes positioning the second instrument so that the instrument tip thereof also engages and lifts a region of the annular recess overhanging lip adjacent the first and second tabs so as to facilitate insertion of the first and second tabs into the primary optic recess by the first instrument.

The step of forming the first and second secondary optic-to-primary optic attachment instruments includes forming each of the instruments having an elongate, slender insertion needle and forming a short, axially extending pin and a pair of short axially extending ears to sides of the pin at a distal end of at least the first instrument needle. The step of forming the first and second secondary-optic-to-primary-optic attachment instruments includes forming each of the first and second instruments having an elongate, slender hollow needle and axially slidingly installing a flexible pin in each of the needles, each of the pins being individually extendable and retractable in the associated needle and includes forming each of the installed pins with a curved distal end region.

The step of forming a thin, elastically deformable secondary optic, preferably includes forming an arcuate recess in the periphery of the secondary optic adjacent each side of each of the first and second tabs so as to facilitate positioning the secondary optic for inserting the first and second tabs into the primary optic annular recess, and preferably includes forming the secondary optic from a silicone or acrylic plastic material.

A secondary optic is provided for attaching to and modifying the optical characteristics of a intraocular lens implanted in a patient's eye, the primary intraocular lens having a primary optic with a narrow annular recess formed into an anterior surface generally parallel to the anterior surface relatively adjacent to a periphery of the primary optic, an entrance to the annular recess having an overhanging lip directed toward an optical axis of the primary optic. The secondary optic has a diameter substantially equal to a primary optic diameter at the annular recess entrance, the secondary optic being formed having at least one insertion tab extending radially outward at a peripheral edge region, the at least one tab being sized for fitting into the primary optic annular recess and having an arcuate recess in the periphery adjacent each side of the at least one tab so as to facilitate positioning the secondary optic for inserting said tab into the annular recess. The at least one tab is formed having a small secondary otic positioning aperture.

Preferably the secondary optic is formed having first and second installation tabs extending radially outward at generally opposite peripheral edge regions, the first and second tabs being sized for fitting into the primary optic recess. An arcuate recess is formed in the secondary optic periphery adjacent each side of each of the first and second tabs so as to facilitate positioning of the secondary optic for inserting the tabs into the annular recess.

The secondary is formed from an elastically deformable material such as silicone and acrylic plastics, and may have a central thickness no greater than about 0.3 mm. The first and second tabs each have a width no greater than about 2 mm and extend beyond the secondary optic peripheral edge a distance of no more than about 0.5 mm.

There is also provided an instrument for installing a peripheral tab of a secondary optic into an annular recess in a primary optic, the secondary optic tab formed having a small positioning aperture. The instrument comprises an elongate, slender needle having a distal end formed having an opposed pair of small, axially-extending, secondary optic holding ears and a slender pin projecting from the needle distal end between the ears, the pin being sized for insertion into the secondary optic tab positioning aperture. Preferably the instrument needle is hollow, the pin being axially slidably installed in the needle to enable selective axial extension and retraction of the pin relative to the needle and the pin being formed having an arcuate distal end region. The pin is then constructed from a shape memory material so as to cause the arcuate distal end region thereof to straighten when the pin is retracted into the needle and to subsequently restore the arcuate distal end region of the pin when the pin is subsequently extended from the needle. The instrument needle is generally cylindrical in shape, the distal end having an oval shape, the needle being constructed to enable a distal end region thereof to be bent at an angle of at least about 10 degrees relative to a remainder of the needle.

Alternatively, an instrument for installing a peripheral tab of a secondary optic into an annular recess in a primary optic, the secondary optic tab being formed having a small positioning aperture. The instrument comprises an elongate, slender needle having a distal end formed having an elongate slender tongue axially projecting therefrom to enable engagement of a peripheral edge region of the primary optic to prevent movement of the primary optic as the secondary optic tab is installed in the primary optic recess. A slender pin projects from the needle distal end, the pin being sized for engaging and lifting an overhanging lip of said primary optic annular recess for facilitating the insertion of said secondary optic tab into said annular recess.

The needle is generally cylindrical in shape, the needle distal end having an oval shape. The needle is constructed to enable a distal end region thereof to be bent at an angle of at least about 10 degrees relative to a remainder of the needle.

The secondary optic and a pair of secondary optic installation instruments form a system for modifying the optical properties of a primary intraocular lens optic, and are used in accordance with the foregoing method of modifying the optical properties of the primary optic.

### BRIEF DESCRIPTION OF THE DRAWINGS

The present invention can be more readily understood by a consideration of the following detailed description when taken in conjunction with the accompanying drawings, in which:

FIG. 1 is a simplified vertical cross sectional drawing of a human eye; FIG. 1a depicting a typical aphakic eye and showing a representative type of posterior chamber IOL implanted in the posterior chamber from which the natural lens has been removed; and FIG. 1b depicting a typical phakic eye and showing a representative type of IOL implanted in the anterior chamber forwardly of the natural lens in the posterior chamber;

FIG. 2 is a perspective drawing of the intraocular lens (IOL) system of the present invention, showing a primary IOL having an optic to which is attached an opposing pair of filament-type fixation elements (haptics), and showing a thin, contact lens-like secondary or supplemental IOL configured for detachable attachment to the primary IOL to provide a predetermined diopter power correction thereto;

FIG. 3 is a transverse cross sectional drawing taken along line 3-3 of FIG. 2 showing the secondary IOL in close proximity to the anterior surface of the primary IOL, but not yet attached thereto;

FIG. 4 is a Fig. similar to FIG. 3, except that the secondary IOL is shown detachably attached to a peripheral recess or channel formed into the anterior surface of the primary IOL optic;

FIG. 5 is a greatly enlarged and detailed transverse cross sectional drawing, in the plane of FIG. 4, of the region in which peripheral regions of the secondary IOL is inserted into the narrow, annular receiving recess or channel formed at an angle in the anterior surface of the primary IOL optic, and showing relative shapes of the edge of the secondary IOL and the mating shape of the receiving recess or channel in the primary IOL;

FIG. 6 is a drawing similar to FIG. 5, showing a variation edge shape of the secondary IOL, the edge having a peripheral, thinned edge region;

FIG. 7 is a plan view of an alternative configuration of a secondary IOL, in accordance with a variation of the preferred embodiment of the invention, showing the edge of the secondary IOL formed having a plurality (three) radially-extending tabs or ears which are configured for insertion into the receiving recess or channel formed in the primary IOL;

FIG. 8 is a transverse cross sectional drawing taken along line 8-8 of FIG. 7, showing grooves formed across a base region of the secondary IOL tabs to facilitate the inserting of the tabs into the primary IOL receiving recess or channel;

FIG. 9 is a sequence of pictorial drawings depicting the primary IOL implanted in the posterior chamber of an aphakic eye and showing the insertion of the secondary IOL into the eye and into the receiving recess or channel formed in the primary IOL; FIG. 9a showing the secondary IOL being inserted by a slender needle through a small corneal incision or hole; FIG. 9b showing the secondary IOL being then inserted through the pupil and into proximity with the anterior surface of the primary IOL; FIG. 9c showing an edge portion of the secondary IOL being inserted or tucked into the receiving recess or channel formed in the anterior surface of the primary IOL optic; and FIG. 9d showing the edge of the secondary IOL fully inserted into the mating primary IOL recess or channel to thereby detachably attach the secondary IOL to the anterior surface of the primary IOL;

FIG. 10 is a perspective drawing of a handling device or apparatus for implanting a secondary IOL into an eye and for extracting (explanting) a secondary IOL from an eye, showing a first, lens implanting/extracting portion comprising a lens holding pin and a retractable shield and showing an associated second portion for receiving the pin and shield portion and for enabling the rotation of the pin and for extending and retracting the shield;

FIG. 11 is a longitudinal cross sectional drawing of the first portion of the handling device of FIG. 10 showing the pin and shield construction thereof, showing a secondary IOL wrapped around a distal end region of the pin and showing the shield extended over the IOL, and further showing attachment proximal end regions of the pin and shield configured for detachably attaching the pin and shield to the second, receiving and control portion;

FIG. 12 is a transverse cross sectional drawing taken along line 12-12 of FIG. 11, showing a secondary IOL wrapped around the distal end of the pin;

FIG. 13 is a longitudinal cross sectional drawing of the second portion of the handling device of FIG. 10, showing a connector region for detachably receiving proximal ends of the pin and shield, and showing a rotary actuator connected for enabling the rotation of the pin when the pin is attached and showing a linear actuator for slidingly extending and retracting the shield when the shield is attached;

FIG. 14 is a perspective drawing of the first portion of the IOL handling device showing the distal end region of the pin in engagement with a representative secondary IOL in preparation for wrapping the IOL around the pin and showing the shield in its retracted position for enabling such IOL wrapping;

FIG. 15 is a transverse cross sectional drawing similar to FIG. 8, showing a variation configuration of a peripheral lip overhanging the primary IOL annular slot which receives a peripheral insertion region of the secondary IOL, the overhanging lip shown having a thickened end region for improved optical characteristics;

FIG. 16 is a plan view of a instrument for attaching a secondary IOL to a primary IOL, showing an ocular insertion portion, an insertion tip portion and a handle portion which includes mechanical controls for operating the insertion tip portion;

FIG. 17 is a transverse cross sectional drawing looking along line 17-17 of FIG. 16 showing control pins for operating the insertion tip portion;

FIG. 18 is a longitudinal cross sectional drawing looking along line 18-18 of FIG. 17 showing a secondary IOL insertion lever axially slidably installed on a fixed, axial post, the lever in an extended position shown in solid lines and in a retracted position shown in broken lines, and showing an end region of a lever operating pin pivotally installed through a finger portion of the lever;

FIG. 19 is a side view of the insertion tip portion (looking along line 9-19 of FIG. 18) showing the offset end region of the lever operating pin, showing a side view of the insertion lever installed on the rigid post and further showing a thumb-shaped secondary IOL edge pushing member;

FIG. 20 is a series of three FIGS. showing the insertion attachment of an edge region of a secondary IOL into a peripheral slot formed on a primary IOL using the insertion tip lever depicted in FIGS. 18 and 19: FIG. 20A showing the lever in its retracted position with the free end of the lever finger portion inserted slightly into the primary IOL slot; FIG. 20B showing the insertion tip lever advanced along the mounting post so that the lever finger portion is fully inserted into the primary IOL slot and with the lip overhanging the slot lifted by the finger portion so the edge region of the secondary IOL can be pushed into the slot; and FIG. 20C showing the lever finger portion flexed away from the primary IOL slot and the secondary IL edge region fully received into the slot;

FIG. 21 is a partially cut-away side view of the insertion tip portion (similar to FIG. 19) showing in solid lines the insertion tip flexed to an extreme left hand position and showing in broken lines the insertion tip portion flexed to an extreme right hand position;

FIG. 22 is a series of three FIGS. showing the insertion attachment of differently located edge region of a secondary IOL into a peripheral slot formed on a primary IOL using the insertion tip of the present instrument: FIG. 22A showing the insertion attachment of an edge region of a secondary IOL into a primary IOL slot in an exemplary situation in which the insertion tip is used in its unflexed condition (i.e., is straight); FIG. 22B showing the insertion attachment of an edge region of a secondary IOL into a primary IOL slot in an exemplary situation in which the insertion tip is used in its fully flexed condition to the left (as depicted in solid lines in FIG. 21); and FIG. 22C showing the insertion attachment of an edge region of a secondary IOL into a primary IOL slot in an exemplary situation in which the insertion tip is used in its fully flexed to the right condition to the left (as depicted in broken lines in FIG. 21);

FIG. 23 is a pictorial drawing showing the implementation of an exemplary manual system used for operating and controlling the insertion tip of the insertion instrument;

FIG. 24 is a plan view of a variation secondary optic for attachment to the optic of a primary intraocular lens implanted in a patient's eye in accordance with the present invention: FIG. 24A showing a secondary optic having first and second insertion tabs at diametrically opposite optic peripheral regions, each tab having a positioning aperture, and showing small arcuate recesses in the secondary optic periphery at the base of each of the tabs, and also showing the secondary optic attached to the optic of an associated primary intraocular lens; and FIG. 24B showing a secondary optic similar to that shown in FIG. 24, except having three insertion tabs, one tab at one secondary optic peripheral region and the other two tabs spaced apart at opposite secondary optic peripheral regions, each tab having a positioning aperture, and showing small arcuate recesses in the secondary optic periphery at the base of each of the three tabs, and also showing the secondary optic attached to the optic of an associated primary intraocular lens;

FIG. 25 is an enlarged view of a representative one of the secondary optic installation tabs of FIG. 24, showing details of the tab and of adjacent peripheral recesses, and further showing details of the associated primary optic;

FIG. 26 is a longitudinal cross sectional view looking along line 26-26 of FIG. 24, showing additional features of the representative secondary optic installation tab as installed in the primary optic annular recess;

FIG. 27 is a perspective drawing of a first instrument for attaching the secondary optic of FIG. 24 to the optic of a primary intraocular lens, showing an elongate, slender hollow needle having a handle and an axially extendable/retractable pin installed in the needle, and an opposed pair of small, secondary optic manipulating/holding ears formed at a distal end of the needle, and showing the pin in its retracted condition and further showing, in phantom lines, the pin in its fully extended, arcuate condition;

FIG. 28 is a partial perspective of a variation secondary optic insertion instrument, similar to the instrument depicted in FIG. 27, for attaching the secondary optic to the optic of a primary intraocular lens, showing a primary optic engaging tongue extending from the distal end of the hollow needle and showing the pin in its extended condition;

FIG. 29 is a pair of plan views corresponding generally to FIG. 24A, showing installation of the secondary optic onto the optic of the primary intraocular lens: FIG. 29A showing the insertion of one of the two secondary optic tabs into the annular recess of the primary optic by two installation instruments inserted into the eye through corresponding ocular incisions; and FIG. 29B showing the insertion of the opposite one of the two secondary optic tabs into the annular recess of the primary optic by the repositioned installation instruments;

FIG. 30 is a pair of corresponding side views showing the insertion of the pin of the instrument of FIG. 27 into the positioning aperture of a representative one of secondary optic tabs and showing the holding of the primary optic by a second one of the instruments of FIG. 27: FIG. 30A showing the positioning of the secondary optic tab at the entrance to the primary optic annular recess and showing the extended pin of the second instrument needle bearing against a peripheral edge region of the primary optic, and showing in phantom lines the distal end of the needle bearing against a peripheral edge region of the primary optic, and FIG. 30B showing the secondary optic advanced so the tab is inserted into the primary optic annular recess by the first instrument needle, while the extended pin of the second instrument needle holds the primary optic in place, and showing in phantom lines the distal end of the needle bearing against a peripheral edge region of the primary optic;

FIG. 31 is a cross sectional view taken along line 31-31 of FIG. 30A showing the insertion of the instrument pin of the instrument of FIG. 27 into the positioning aperture of a representative one of the secondary optic tabs, and showing the pair of ears at the distal end of the instrument needle bridging the tab to assist in the positioning of the secondary optic; and

FIG. 32 presents three views showing in an alternative the manner in which the extended pin of the instrument needle of FIG. 28 engages the overhanging lip of the primary optic annular recess: FIG. 32A showing the extended pin of the instrument needle positioned to lift the overhanging lip while the instrument needle tongue is positioned against a peripheral edge region of the primary optic, FIG. 32B showing the primary optic recess lip lifted by the extended pin of the instrument needle to open the recess while the instrument needle tongue bears against the primary optic peripheral edge region, and FIG. 31C showing the distal end of the instrument needle of FIG. 27 having moved the secondary optic so the representative tab is inserted into the opened primary optic annular recess.

In the various FIGS., the same elements and features are given the same reference numbers, and similar elements and features are given the same reference number as the original element or feature followed by the letter “a”, “b”, “c” and so forth as appropriate.

### DESCRIPTION OF THE PREFERRED EMBODIMENT

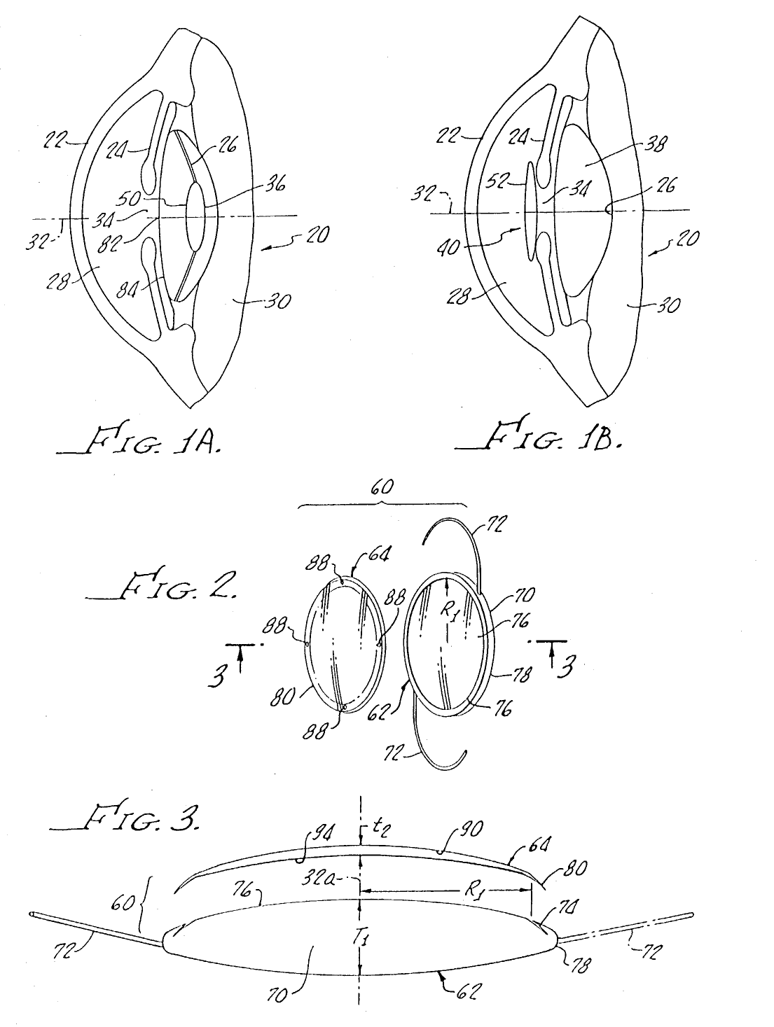

There is shown in FIGS. 1a and 1b, principally for reference purposes, a simplified cross sectional view of forward regions of a representative human eye 20. Comprising eye 20 are a cornea 22, an iris 24, a posterior chamber 26, and an anterior chamber 28. A vitreous humor 30 is contained in eye 20 rearwardly of posterior chamber 26. Eye 20 is further shown having an optical axis 32 that passes through cornea 22, an iris opening 34, and anterior and posterior chambers 28 and 26.

In particular, FIG. 1a depicts eye 20 as an aphakic eye in which a posterior chamber IOL 36 is implanted in posterior chamber 26 from which the natural lens has been removed. FIG. 1b, on the other hand, depicts eye 20 as a phakic eye with a natural lens 38 shown remaining in its normal position in posterior chamber 26. FIG. 1b additionally shows an IOL 40 implanted in the anterior chamber 28 of eye 20 forward of natural lens 38.

As further shown in FIG. 1a optical axis 32 of eye 20 passes through the center of an optic 50 of posterior chamber IOL 36; and as shown in FIG 1b the optical axis passes through the centers of both an optic 52 of anterior chamber IOL 40 and natural lens 38.

The spherical, cylindrical and/or add power of an IOL to be implanted in a patient's eye is selected by the ophthalmologist in the case of an aphakic eye to restore as closely as possible normal vision and in the case of a phakic eye to correct the patient's vision.

It will be appreciated that determining the exactly correct power of the IOL to be implanted is often difficult because of such factors as the current condition of the patient's eye and effects of the surgical procedure. Consequently, an IOL diopter with a small add power may be selected, even if not actually needed.

Moreover, even if an IOL with an optimum diopter power is implanted, the condition of the patient's eye may deteriorate, for example as the patient ages, such that the IOL diopter is no longer optimum. Furthermore, the actual axial position of the implanted IOL may be slightly different than the intended axial position, thereby introducing an error in the “effective” power of the IOL. In addition, a non-spherical, i.e., cylindrical, cornea can reduce image quality further.

Thus, for whatever reason, the initially selected power of the implanted IOL may not initially, or at some future time, provide the optimum vision or intended vision correction for the patient. To provide the needed or desired power correction, spectacles or contact lenses could be provided but that would tend to obviate the advantages provided by the implanted IOL,

On the other hand, the explanting of the non-optimum IOL and the implanting of another IOL in its place would involve significant surgical intervention with no guarantee that the new IOL would provide the desired correction.

The in situ correction of the power of an already implanted IOL is, therefore, highly desirable in many circumstances, and is the focus of the present invention.

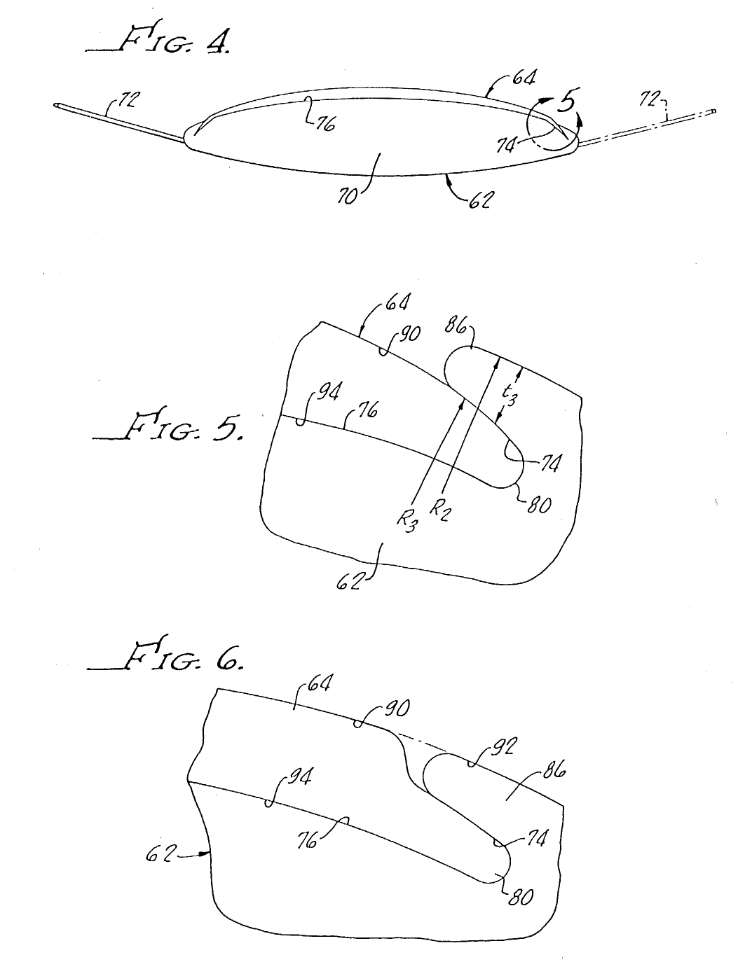

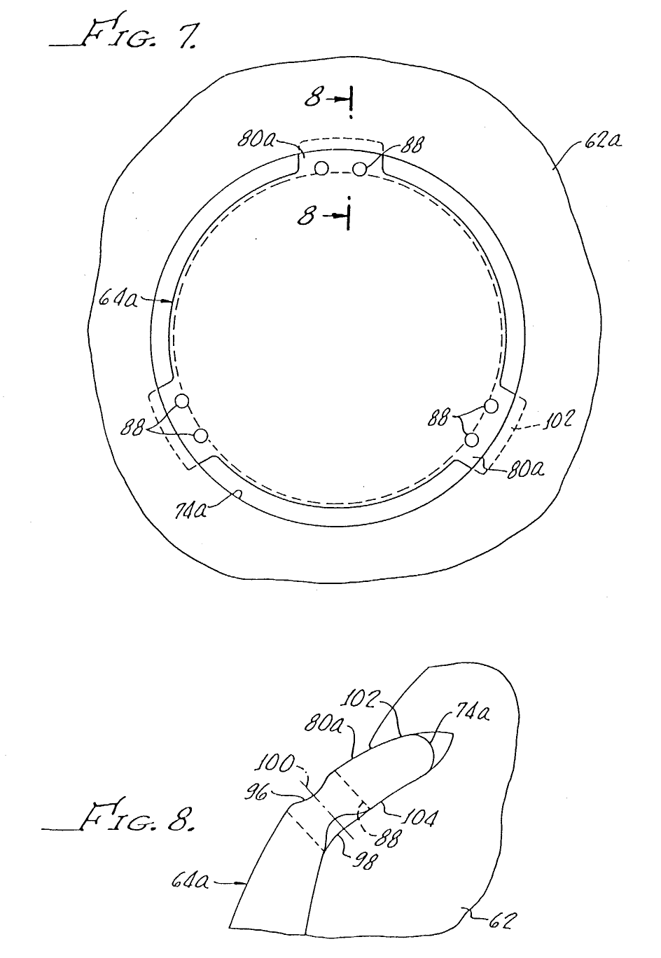

To this end, and as best shown in FIGS. 2-4, the present invention comprises an IOL system 60 that includes a primary IOL 62 and a secondary IOL 64 which are configured so the secondary IOL can be readily attached to and detached from the implanted primary IOL, with minimal surgical intervention to the patient, and without disrupting the position of the primary IOL in the eye.

Primary IOL 62 is thus configured to provide the principal vision correction and may be provided in about eighty percent (80%) of cases in the range of between about −1.0 and about +1.0 diopters. The error is most significant for short (i.e., ≦about 20 mm) or long (i.e., ≧about 25 mm) eyes. In any event, primary IOL 62 may be provided with a spherical power of between about −10 and +35 diopters; a cylinder power of between about 0.0 and about +10 diopters; and/or an add power of between about 0 and about +4.0 diopters.

Since secondary IOL 64 is configured to provide only a small diopter correction to primary IOL 62, it may be provided in the diopter range of between about +2.5 and about −2.5. In any event, secondary IOL 64 may be provided with a spherical power of between about −3.0 and +3.0 diopters; a cylinder power of between about −5.0 and about +5.0 diopters; and/or an add power of between about +1.0 and about +4.0 diopters.

Primary IOL 62 has a center optic thickness, t₁, which may be in the usual range of between about 0.6 mm and about 1.5 mm, and secondary IOL 64 may have a center optic thickness, t₂, in the range of only about 0.1 mm and about 0.2 mm.

With no limitation being intended or implied, primary IOL 62 is disclosed herein as being a posterior chamber IOL (corresponding generally to above mentioned posterior chamber IOL 36). It is, however, to be understood that primary IOL 62 may alternatively be configured for optic 70 thereof to be implanted in the patient's anterior chamber 28 (thereby corresponding generally to above mentioned anterior chamber IOL 40). In addition, again with no limitation being intended or implied, primary IOL 62 is disclosed as a non-plate-type IOL.

Thus, as shown in FIGS. 2-4, primary IOL 62, which may be a one piece rigid IOL (e.g., a PMMA IOL) or a three piece foldable IOL (e.g. a silicone or soft acrylic IOL), comprises an optic or optic region 70 to which is attached an opposing pair of positioning elements, called haptics, 72. These haptics 72 may consist of slender, springy filaments, as shown, or may comprise flexible plates (not shown), both as are common in the IOL field.

To enable detachable attachment of secondary IOL 64 to primary IOL 62, primary IOL optic 70 is formed having a narrow recess or groove or channel 74 formed into an optic anterior surface 76 adjacent a peripheral optic edge 78.

Primary IOL annular recess or channel 74, is sized for receiving and releasably retaining a peripheral edge region 80 of secondary IOL 64, which can readily be inserted by an ophthalmic surgeon into the recess or channel with minimal invasive surgery and without changing the position of primary IOL 62, in the manner described below. Recess or channel 74 may have a width, w, of about 0.5 to about 2.0 mm and a depth, d, of about 0.2 to about 0.9 mm.

Although recess or channel 74 is shown in FIG. 2 as extending completely around optic 70 for receiving complete peripheral edge region 80 of secondary IOL 64, it is to be understood that recess or channel 74 may alternatively extend only partially around the optic, and may be provided in one or more angular segments for receiving only portions of the secondary IOL edge.

To avoid or reduce possible visual effects due to recess or channel 74, the recess or channel is preferably located at a radial distance, R₁, from an optical axis 32a that causes the recess or channel to be outside the patient's pupil as defined by iris 24.

For an aphakic eye in which primary IOL 62 is implanted in posterior chamber 26, a central, circular opening 82 (capsularexus) is typically surgically cut in an anterior wall 84 of the posterior chamber (FIG. 1a) during the phacoemulsification procedure. The size of opening 82 is ordinarily about 5 to about 6 mm, so that the removal of the natural lens is facilitated and so that anterior wall 84 does not interfere with light transmission to the implanted IOL. Consequently, the primary IOL recess or channel radius, R₁, is preferably selected to be somewhat greater than about 3 mm so that recess or channel 74 is located radially outwardly of anterior wall opening 82, and out of the path of light entering eye 20.

Nevertheless, there still exists some possibility that glancing rays of light may pass through the region of recess or channel 74 even for a posterior chamber implanted primary IOL 62 and may be greater when the primary IOL is implanted in anterior chamber 28. Therefore, edge 80 of secondary IOL 64 and recess or channel 74 of primary IOL 62 are preferably constructed as shown in FIG. 5 so the edge fits closely into the recess or channel to thereby eliminate or minimize any interior IOL-air interfaces. Moreover, it is preferable that an external radius of curvature, R₂, of a lip portion 86 of primary IOL 62 defining recess or channel 74 be made the same as the internal radius of curvature, R₃, of the lip portion, i.e., that the lip portion have a constant thickness, t₃.

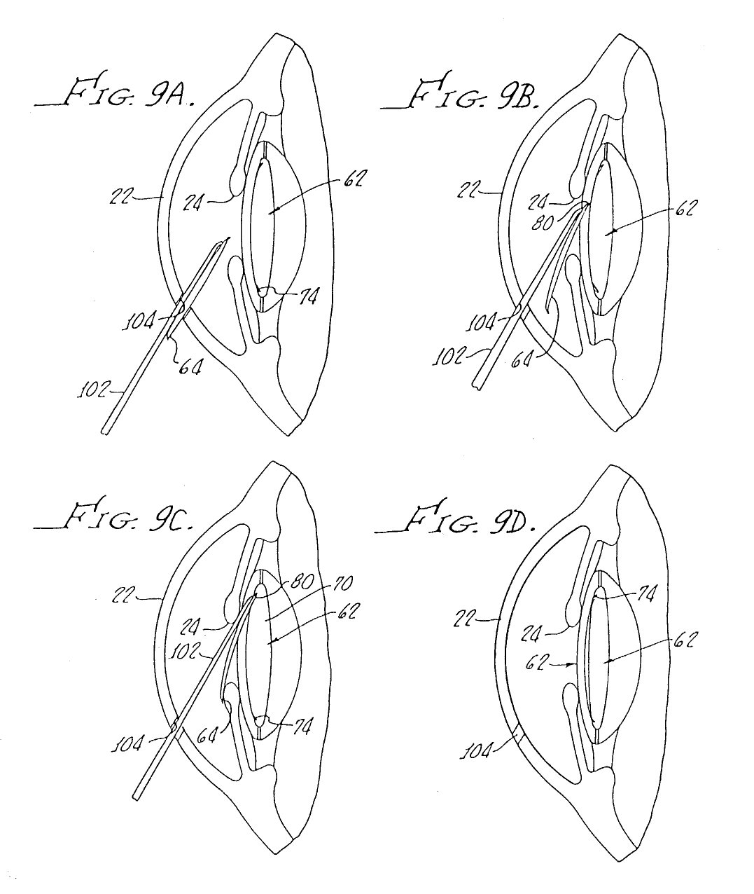

In the variation depicted in FIG. 6, some optical improvement may be achieved by bulging secondary IOL 64 adjacent edge region 80 so that an anterior surface region 90 of the secondary IOL is substantially in line with or substantially continuous with an exterior surface 92 of primary IOL lip portion 86.

Power of primary IOL 62 is dependent upon anterior and posterior surface curvature, preferably a fixed curvature of anterior surface 76 is provided to maintain a constant arc length for all diopter powers or at least constant for each broad range of diopter power.

Otherwise, except for recess or channel 74, primary IOL is preferably similar to a conventional IOL, and is manufactured by known IOL fabrication processes and may be made of any approved material, size, shape and power.

Secondary IOL 64 is preferably formed having at least one small hole 88 (four such holes being shown in FIG. 2 at ninety-degree intervals) formed relatively adjacent edge 80. By means of hole or holes 88 secondary IOL 64 can easily be positioned in the patients eye 20 and inserting of the edge into primary IOL recess or channel 74 is facilitated.

Secondary IOL 64 is made of a thin, elastically-deformable (flexible), biocompatible material and has a convex anterior surface 90 and a concave posterior surface 94 (FIG. 2), to which cylinder correction may be applied. When attached to anterior surface 76 of primary IOL 62, secondary IOL 64 will generally lie along such anterior surface, but is not held or clamped thereto; consequently, any corrective power formed on posterior surface 94 will not be adversely affected.

Although in some respects secondary IOL 64 resembles a conventional contact lens, one principal distinction is that whereas a contact lens has a diameter of about 9 mm for an RGP (rigid, gas permeable) contact lens and a diameter of about 13 to 14 mm for a soft contact lens, secondary IOL 64 has an overall dimension not greater than about 7 mm, (i.e., less than the diameter of primary IOL optic 70). Consequently secondary IOL 64 is best suited for construction by conventional IOL manufacturing processes.

Variation Secondary IOL of FIGS. 7-9:

As an alternative to secondary IOL 64 being formed having a completely circular edge 80, all of which is inserted, upon attachment to primary IOL recess or channel 74, as described above, the secondary IOL may more preferably be formed having a discontinuous edge resulting in a plurality of projecting edge regions in order to facilitate the attachment of the secondary IOL to primary IOL 62a.

Thus, as depicted in FIGS. 7, a variation secondary IOL 64a is formed having three equally spaced apart edge tabs or ears 80a, each of which is configured for being inserted into a corresponding region of the primary IOL recess or channel. As shown, each such tab 80a is about 25 to about 30 degrees wide. Alternatively, only one such tab 80a may be provided, or any number of such tabs may be provided.

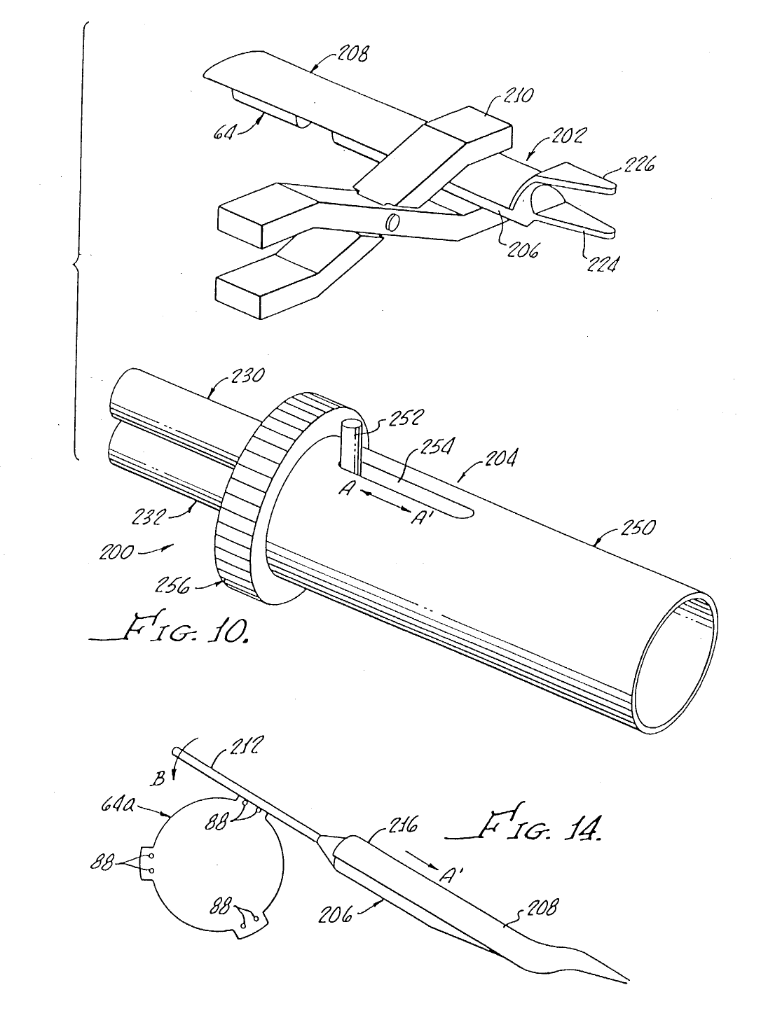

Tabs 80a may, as shown in FIG. 8 for a representative one of the tabs, be formed having a shallow upper transverse groove 96 and/or a shallow lower transverse groove 98 (both grooves being shown by way of example). Groove 96 and/or groove 98 define a bend or hinge line 100 in tabs 80a thereby facilitating the insertion of the tabs (or tab) into recess or channel 74a of primary IOL 62a.

By way of further example, recess or channel 74a of primary IOL 62a may be formed having a concave upper, inner surface 102 and a concave lower, inner surface region 104. Consequently, light entering recess or channel 74a is scattered away from the image focal point of the patient's eye. As above described for recess or channel 74 of primary IOL 62, recess or channel 74a may extend only partially around the primary IOL optic,

In addition, each of tabs 80a is preferably formed having at least one positioning and manipulating holes 88 along bend line 100 (two of such holes being shown in each tab 80a in FIG. 7).

Secondary IOL 64a is detachably attached to primary IOL 62a by tabs (or only a single tab) 80a or, as disclosed above, by edge region 80, and there no pressure applied by the secondary IOL against the primary IOL. Otherwise, secondary IOL 64a is preferably the same as secondary IOL 64 and is constructed in the same manner.

Because of the described method of detachably attaching secondary IOL 64 and 64a to primary IOL 62 and 62a, respectively (i.e., the secondary IOL is only supported in place without any clamping or fastening) the back (posterior) surface shape of secondary IOL 64 or 64a is preserved. As an example, the posterior surface of the secondary IOL can be used for cylinder correction and the anterior surface of the secondary IOL can be used for spherical correction and multifocal power for presbyopia correction.

Implanting and Attachment of Secondary IOL (FIG. 9):

FIG. 9 illustrates a technique for implanting a thin, foldable secondary IOL represented by secondary IOL 64 into eye 20 and into detachable attachment to a primary IOL represented by primary IOL 62 of the present invention (that is, an IOL formed having an edge receiving recess or channel 74). For purposes of describing the secondary IOL implanting and attachment process, primary IOL 62 is depicted as an IOL that has been previously implanted in posterior chamber 26.

FIG. 9 depicts, for illustrative purposes and with no limitation being thereby intended or implied, the implanting of secondary IOL 64 and attachment thereof to a posterior chamber-located primary IOL 62 in an aphakic eye. It is, however, to be understood that the technique applies as well to the implanting and the attachment of secondary 64a to primary IOL 62a. Moreover, the technique applies equally to the implanting and attachment of secondary IOL 64 or 64a to primary IOL 62 or 62a of the anterior chamber type previously implanted in anterior chamber 28.

Thus, FIG. 9a depicts secondary IOL 64 being inserted by a slender needle-like implement 102 through a very small hole 104 in cornea 22 (or in the sclera), for example punched by the implement. Since secondary IOL 64 is thin and flexible, it can be inserted through a corneal or sclera hole no larger than about 2.0 to 2.5 mm. The end of implement 102 can advantageously be inserted through IOL edge hole 88.

In FIG. 9b, secondary IOL 64 is shown inserted entirely through corneal hole 104 and partially through the patient's pupil, and into proximity with primary IOL 62. FIG. 9c depicts upper regions of edge 80 of secondary IOL 64 being inserted or tucked into primary IOL recess or channel 74. In turn, FIG. 9d depicts secondary IOL edge 80 entirely inserted into primary IOL recess or channel 74 with the secondary IOL (actually, posterior surface 94 thereof) in contact with primary IOL anterior surface 64, and with implement 102 having been removed from eye 20.

In the event replacement of secondary IOL 64 becomes desirable or necessary, for example, to implant a secondary IOL of a different power (spherical, cylinder or add), the detaching of the secondary IOL from primary IOL 62 and removal of the secondary IOL from eye 20 is easily accomplished by reversing the implanting procedure described above. Alternatively, after detachment of secondary IOL 64 from primary IOL 64, the secondary IOL can be cut or torn into several pieces for easier removal from the eye.

In the described manner, secondary IOL 64 can be replaced by a different secondary IOL of different power which can easily, and with minimal patient trauma, be implanted and attached to primary IOL 62 if an attached secondary IOL no longer provides the desired correction power.

Presbyopic correction of a dynamic phakic eye may, as an illustration, require the implanting of a two or more successive secondary IOLs of progressively increased add during a period of about 10 to 15 years as the patient's eyes change with increased age. As a nonlimiting example, a first secondary IOL implant of about 1.25 diopter power may be required for early presbyopes. A second secondary IOL implant of about 1.75 diopter power may subsequently be required in about 5 to 7 years after the first implant. A third secondary IOL implant of about 2.25 diopter power may subsequently be required in another 5 to 7 years.

It is further to be understood that primary IOL 62 or 62a (or equivalent) as above described and of any desired diopter may typically be manufactured independently of the manufacture of secondary IOL 64 or 64a (or the equivalent) and will usually be separately implanted in a phakic or aphakic eye. The desirability of implanting the secondary IOL and detachably attaching it to primary IOL may occur just after the primary IOL is implanted and during the same surgical procedure (to provide an initial correction, sometime later as noted above, or possibly not at all. Nevertheless, the primary IOL would be appropriately configured for the attachment of a secondary IOL of any desired diopter, when the necessity or desirability of doing so arises.

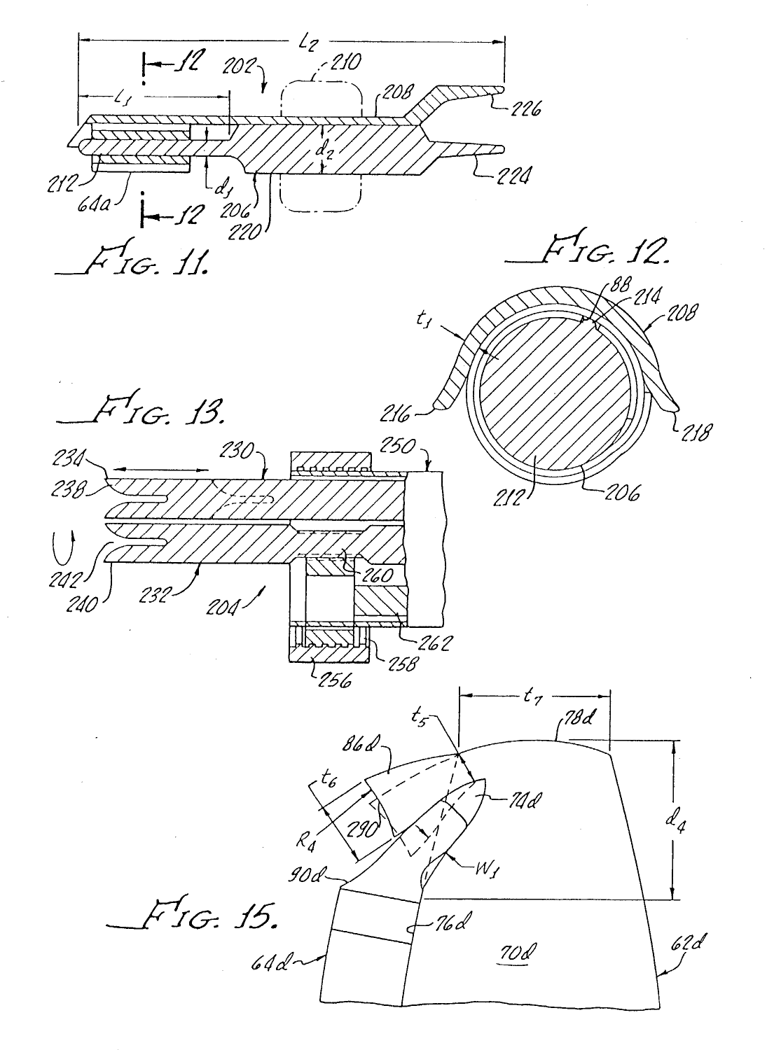

The Secondary IOL Handling Apparatus of FIGS. 10-14:

Although a conventional pin-type instrument 104 may be used for implanting and extracting (explanting) secondary IOL 64 (and 64a) as has been described above relative to FIG. 9, a specialized secondary IOL handling device or apparatus 200 (FIGS. 10-14) is useful for handling (i.e., implanting and extracting) the secondary IOL, especially in the close confines of the anterior chamber of the eye.

Shown comprising handling device 200 are a first, pin and shield portion 202 and a second, pin and shield receiving and controlling portion 204 (FIG. 10). Because of concern over the transmission of body fluid-borne diseases, such as AIDS and hepatitis, first portion 202 may be made disposable after a single use. In contrast, second portion 204, which is not expected to come in contact with body fluids, may be made reusable.

Shown comprising first portion 202 are an elongate, rigid pin 206 and a flexible shield 208 (FIGS. 10 and 11). Shown in FIG. 10 is a conventional small clamp 210 that is used to hold pin 206 and shield 208 together until they are installed in second portion 204.

As seen from FIG. 11, an elongate, round distal end region 212 of pin 206 is formed having a uniform diameter, d₁, that is only about 0.5 to about 0.8 mm. Length, L₁, of pin distal end region 212 is preferably about 8 to 10 mm. Pin 206 may be constructed of a surgical grade of stainless steel or titanium.

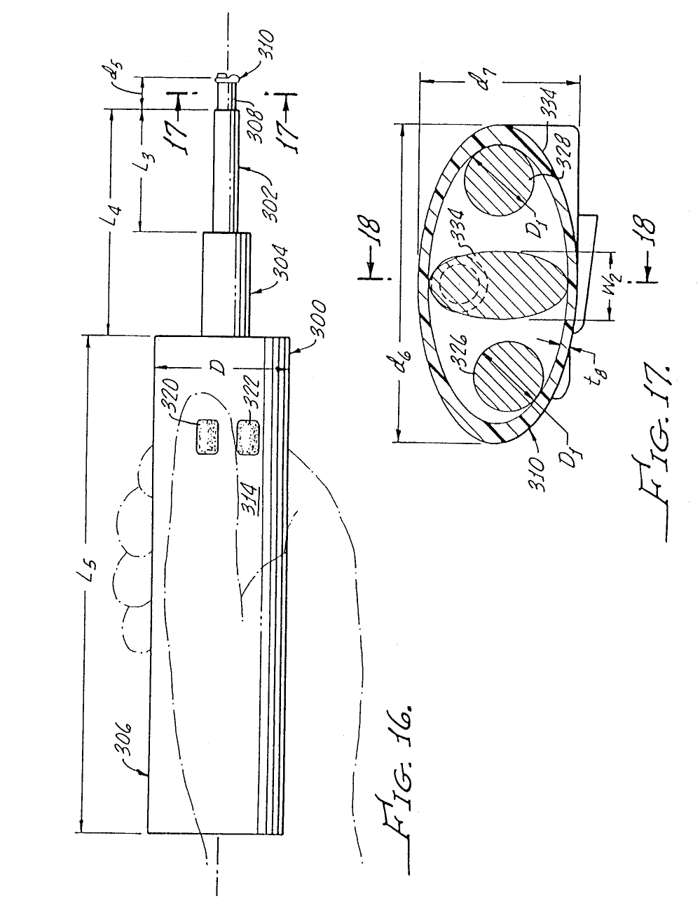

A small secondary IOL pin-like engaging projection 214 formed on pin distal end region 212 in a generally central location thereof is sized to fit into the or one of the holes 88 of representative secondary IOL 64a that (FIG. 12). As shown, secondary IOL 64a (or 64) is then wrapped closely around distal end region 212 in preparation for implanting in an eye and for attachment to primary IOL 62 (or 62a) in the above-described manner. As a non-limiting example, for a pin end region diameter, d₁, of about 0.8 mm, and a secondary IOL diameter of between about 5.0 mm to 6.0 mm, secondary IOL 64a (or 64) will wrap around pin end region 212 about twice (as depicted in FIGS. 11 and 12).

Shield 208, which is constructed from an elastomeric material, such as silicone or an acrylic material, is constructed having at least a distal end region 214 that is semi-cylindrical in shape (FIG. 12). Shield end region 216, which is sized to slide axially, in a close fitting relationship, over a secondary IOL 64 (or 64a) wrapped around pin end region 212 to thereby keep the IOL wrapped around the pin end region, preferably has a thickness, t₄, of about 0.3 mm. Side edges 218 of shield end region 216 are preferably slightly flared out as depicted in FIG. 12 to provide for sealing the ocular incision (not shown) made for implanting or extracting the secondary IOL.

A central region 220 of pin 206 is made having a diameter, d₂, that is about three times the distal end region diameter, d₁. A proximal end 224 of pin 206 is tapered in an elongate cone shape (FIGS. 10 and 11). A proximal end 226 of shield 208 is similarly tapered in an elongate cone shape. Overall length, L₂, of portion 202 may be from about 15 mm to about 25 mm.

Receiving and control portion 204, as shown in FIG. 13, comprises a linear actuator or means 230 configured for imparting linear, axial movement to shield 208 and a rotary actuator or means 232 configured for imparting rotational movement to pin 206. To this end, a distal end 234 of linear actuator 230 is formed having a conically tapered socket 236 sized to receive proximal end 226 of shield 208 in frictionally locking engagement. Similarly, a distal end 238 of rotary actuator 232 is formed having a conically tapered socket 240 sized to receive proximal end 224 of shield 206 in frictionally locking engagement.

Proximal ends of linear actuator 230 and rotary actuator 232 are retained in a handle-like housing 250. As shown in FIG. 10, linear actuator 230 is moved for advancing and retracting in an axial direction (Arrow A-A′) by a lever 252 that protrudes through an elongate slot 254 in housing 250. Rotary actuator 232 is rotated in either rotational direction (Arrow B-B′) by a thumb wheel 256 that is interconnected with the rotary actuator by a drive gear 258 that engages a gear 260 formed on the rotary actuator (FIG. 13). Drive gear 258 is mounted on a shaft 262 that is, in turn, mounted in housing 250.

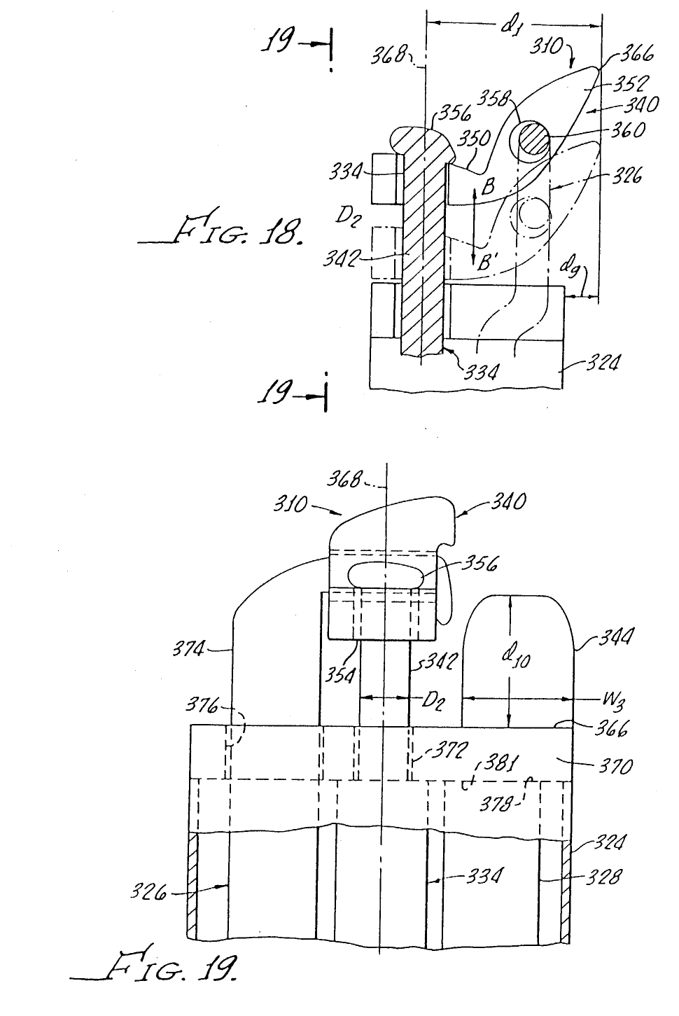

In use, pin proximal end 224 is frictionally connected or coupled to socket 242 of rotary actuator 232 and shield proximal end 226 is frictionally connected or coupled to socket 238 of linear actuator 230. Then, rotational movement of rotary actuator 232, by thumb wheel 256, causes rotation of pin 206. Depending upon the direction of rotation of pin 206, an unwrapped secondary IOL (for example, IOL 62a) engaged by projection 214 is can be wrapped around pin distal end region 212 (as depicted in FIG. 14) for implanting into a patient's eye or a secondary IOL wrapped around the pin distal end can be unwrapped, for example, after the IOL has been inserted into a patient's eye in proximity to an implanted primary IOL.

While a secondary IOL is being wrapped around or unwrapped from pin distal end region 212, shield 208 is retracted by operation of lever 252 so that distal end region 214 is out of the way (FIG. 14). After a secondary IOL has been wrapped around pin distal end region 212, shield 208 is advanced by lever 252 so that shield distal end region 214 extends over the wrapped IOL to keep it wrapped.

As device 200 implants a secondary IOL wrapped around pin region 212 through an ocular incision, shield end region 216, which had been extended to cover the wrapped IOL, is retracted so as to stay in and seal the incision.

IOL handling device 200 provides good control over the secondary IOL during its implanting in or extraction from a patient's eye, as is especially needed, for example, when applying the secondary IOL to or removing it from a primary IOL optic located in the anterior chamber of the eye where there exists only minimal clearance between such optic and the sensitive endothelial surface of the cornea.

Importantly, device 200 permits manipulation of the secondary IOL with one hand. A small manipulator-probe (not shown) can be employed by the other hand to assist in unwrapping an inserted secondary IOL and placement thereof into recess 74 or 74a of the implanted primary IOL.

Primary IOL Recess or Channel Variation of FIG. 15:

There is depicted in FIG. 15, a variation recess or channel 74d, formed in a primary IOL 62d for receiving an edge region 80d of a secondary IOL 64d. Partially defining recess or channel 74d is a wedge-shaped, peripheral lip portion 86d that overhangs the recess or channel and that extends for the annular arc length of the recess or channel.

The upwardly flexed position of lip portion 86d is shown in solid lines in FIG. 15 for the situation in which secondary IOL edge region 80d is received (i.e., installed) in primary IOL recess or channel 74d. In contrast, lip portion 86d is depicted in broken lines for the for the situation in which no secondary IOL edge region 80d is received (i.e., installed) in primary IOL recess or channel 74d.

Lip portion 86d, as depicted in FIG. 15, is wedge shaped and may have a base thickness, t₅, of about 0.15 mm and a thicker free end thickness, t₆, of about 0.22 mm. Thicknesses t₅ and t₆ are, however, dependant upon the elasticity of primary IOL 62d and are selected to provide flexing of lip portion 86d without shifting of the primary IOL. Recess or channel 74d preferably has an undeflected width, w₁, of about 0.10 mm. A concave end surface 290 of lip portion 86d has a preferred radius, R₄, of about 15 mm. Recess or channel 74d starts at a distance, d₄, of about 0.50 mm from a primary IOL optic edge 78d.

Advantages of the above-described wedge-shaped configuration of lip portion 86d are the effective refraction of any incident light away from the IOL focal point and an increased elastic restoring force for retaining the associated secondary IOL edge region 80d in recess or channel 74d after the insertion of the edge region in the recess or channel.

If an anterior surface 76d of primary IOL 62d were extended to edge 78d of optic 70d (as depicted by a broken line in FIG. 15, the optic edge would preferably have a edge thickness, t₇, of about 0.40 mm.

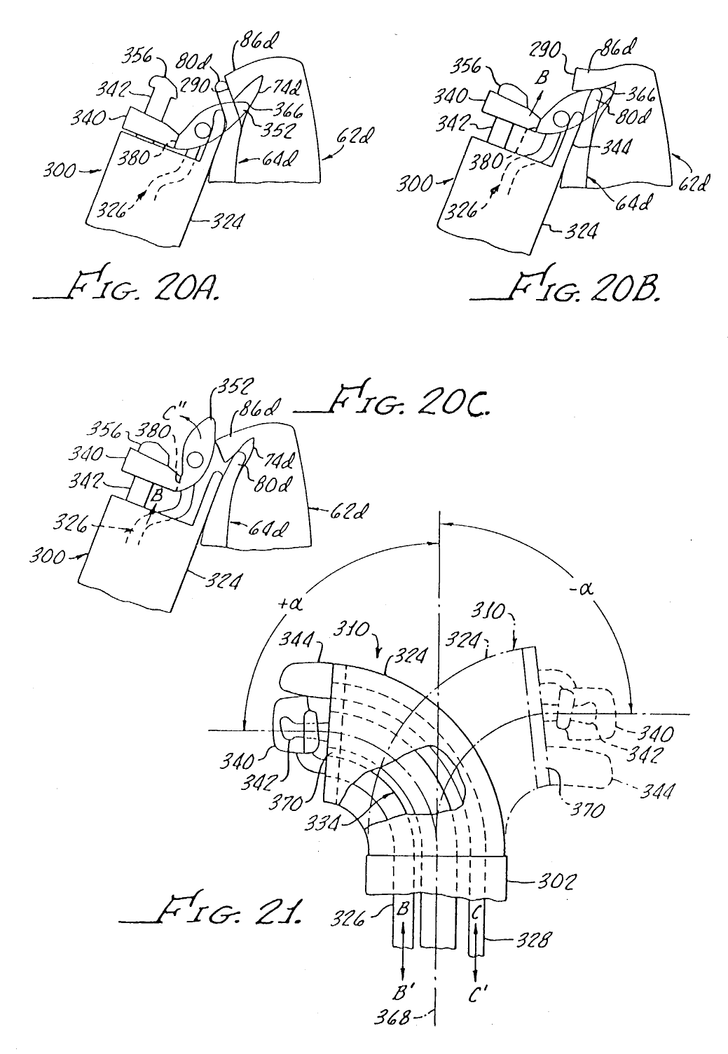

Secondary-Primary IOL Attachment Instrument, FIGS. 16-23:

FIG. 16 depicts a new and novel hand-held instrument or implement 300 that can advantageously be used for attaching a secondary IOL (such as secondary IOLs 64-64d) to a primary IOL (such as primary IOLs 62-62d). As more particularly described below, instrument 300 is effective for inserting a secondary IOL edge region (such as edge regions 80-80d) into a primary IOL recess or channel (such as recesses or channels 74-74d).

Shown generally comprising instrument 300 are an elongate, slender ocular insertion portion 302, a transition portion 304 and a handle portion 306. Projecting axially from an open distal end 308 of ocular insertion portion 302 is a operating tip assembly 310 (more particularly described below).

Operating tip assembly 310 preferably extends a distance, d₅, of about 3 mm beyond distal open end 308 of insertion portion 302 which, in turn, has a preferred length, L₃, of about 12 mm. Further, ocular insertion portion 302 and transition portion 304 preferably have a combined length, L₄, of about 27 mm. Handle portion 306 has a preferred length, L₅, of about 150 mm and a preferred diameter, D, of about 25 mm.

Projecting outwardly from a side surface 314 of handle portion 306 are first and second thumb wheels 320 and 322 which form part of a operating tip assembly operating and control system (not shown in FIG. 16).

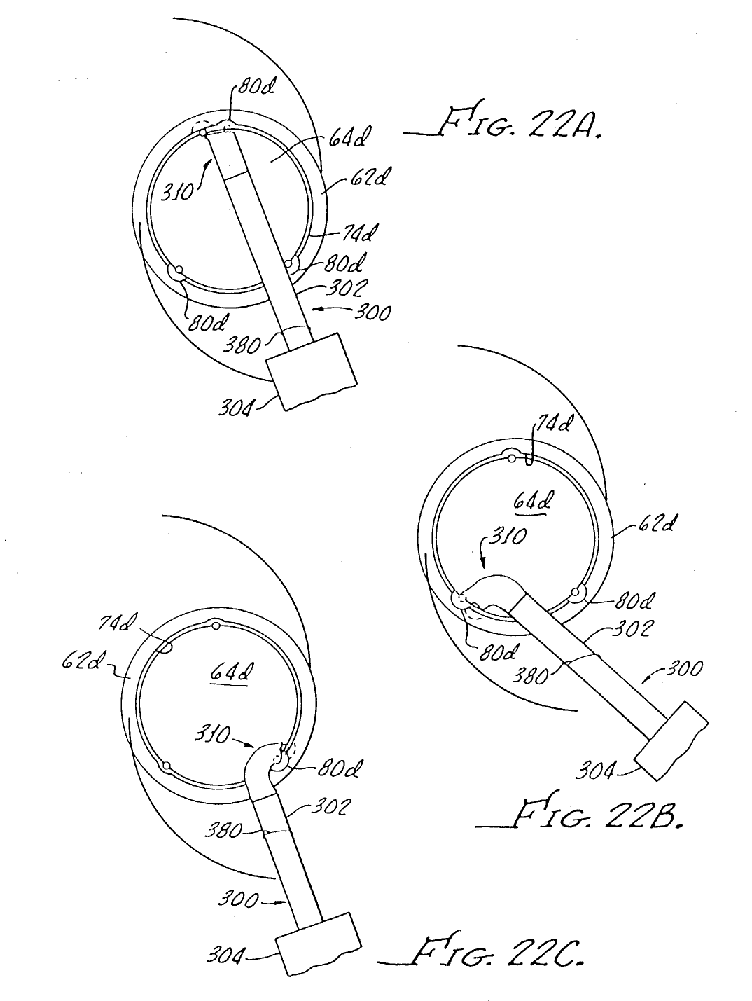

A transverse cross section of operating tip assembly 310 is shown in FIG. 17. Depicted is an elliptically shaped or oval exterior protective sleeve or shroud 324 having a major outside dimension, d₆, of about 2.0 mm, a minor outside dimension d₇, of about 1.0 mm and a wall thickness, t₈ of about 0.1 mm. Sleeve 324 is preferably constructed of an elastomeric biocompatible material, such as a silicone or an acrylic material.

Installed axially through sleeve 324 are respective first and second elongate, slender, axially-movable cylindrical pins or rods 326 and 328 which form part of an operating tip assembly operating and control system (more particularly described below). Installed along a central plane 332 of protective sleeve 324 is a third, elongate, slender, axially fixed, elliptically shaped (in transverse cross section) pin or rod 334. Each of first and second pins or rods 326 and 328 has a cross sectional diameter, D₁, of about 0.5 mm, which is about equal to a width, w₂, of third pin 334. All of pins 326, 328 and 334 are preferably formed of a biocompatible material, such as stainless steel. Pins 326 and 328 are relatively flexible due to their very small diameter.

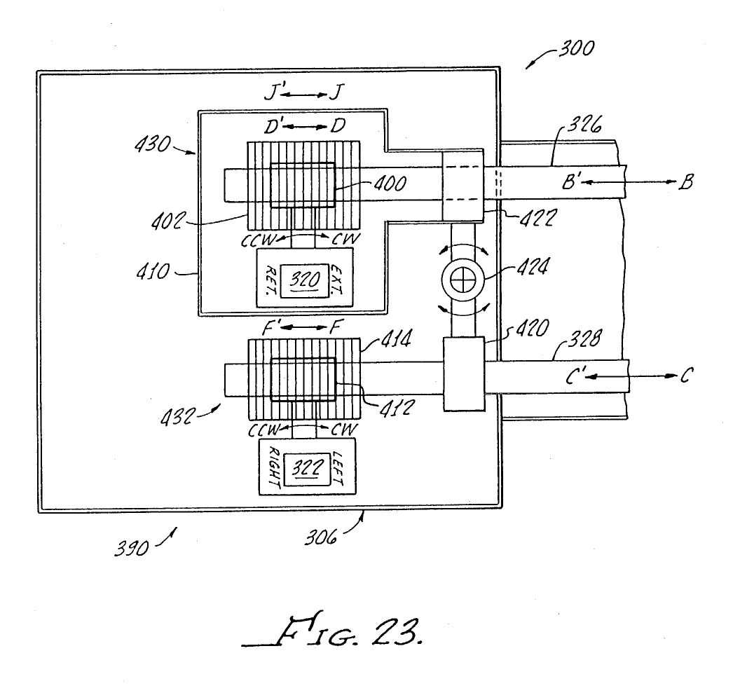

FIGS. 18 and 19 show features of operating tip assembly 310 which principally comprises a movable lever 340, a fixed lever-mounting post 342 (which forms a distal end region of third pin or rod 334) and a secondary IOL edge pushing element 344. Movable lever 340 is formed having a body portion 350 and a sidewardly and a relatively slender, axially-forwardly angled primary IOL recess-engaging finger portion 352.

Lever body portion 350 is formed having an axial aperture 354 by which lever 340 is slidably installed on post 342 for limited axial movement (direction of Arrows B-B′) of about 0.5 mm between a fully extended position (shown in solid lines in FIGS. 18 and 19) and a fully retracted position (shown in broken lines in FIG. 18).

The axial extended position of lever 340 (in the direction of Arrow B), by axial movement of associated first pin or rod 326, is limited by a flared head 356 formed at the distal end of post 342. Post 342 preferably has a diameter, D₂, of about 0.3 mm.

Lever finger portion 352 is formed having a transverse aperture 358 (FIG. 18) through which a sidewardly stepped and right angle bent distal end region 360 of first pin 326 is pivotally installed. A flared head 362 (FIG. 19) at the end of end region 360 retains the end region in lever finger portion aperture 358.

A tapered distal tip 366 of lever finger portion 352 extends sidewardly from a centerline 368 a distance, d₈, of about 0.8 mm and sidewardly beyond sleeve 324 a distance, d₉, of about 0.2 mm (FIG. 18).

Lever 340 is constructed from a biocompatible material, preferably, but not necessarily, of an elastomeric material such as a silicone or an acrylic material.

Secondary edge pushing element 344, which is generally thumb-shaped, has a thickness (not shown) of about 0.15 mm, a preferred base width, w₃, of about 0.6 mm and extends an axial distance, d₁₀, of about 0.75 mm beyond an open distal end of sleeve 324 (FIG. 19).

As shown in FIGS. 18 and 19, a transverse member 370 is installed at sleeve open end 366. Post 342 (which forms the distal end region of third pin or rod 334) extends axially through an aperture 372 in transverse member 370 and a forward region 374 of first pin or rod 326 extends axially through an aperture 376 in the transverse member. Further, a distal end surface 378 of second pin or rod 328 is fixed to an under surface 380 of transverse member 370 or is otherwise connected to the transverse member.

FIG. 20 depicts in diagrammatic form the use of instrument 300, in particular, the operation of operating tip lever 340, in the insertion of a secondary IOL edge region 80d into primary IOL recess or channel 74d in accordance, by way of example only, with FIG. 15. It is, however, to be understood that the use of instrument 300 is not limited to insertion of secondary IOL edge regions in primary IOLs having the recess lip configuration depicted in FIG. 15, but is applicable to other types of recess lip configurations, including those depicted in FIGS. 5, 6 and 8.

FIG. 20A depicts instrument 300 positioned so that above-described lever finger tip 366 is in the entrance to primary IOL recess or channel 74d, under the free end of primary IOL lip portion 86d. Secondary IOL 64d is separately positioned so that its outwardly flexed edge region 80d lies along lip portion end surface 290.

Instrument lever 340 is then extended (advanced), as depicted in FIG. 20B, in the direction of Arrow B by first pin 326, thereby pushing lever finger tip 366 into primary IOL recess or channel 74d with lever finger portion 352 lifting lip portion 86d to open the recess or channel. As recess or channel 74d is opened by lever finger 352 instrument pushing element 344 pushes or nudges secondary IOL edge region 80d into the opening recess or channel.

An additional operational step depicted in FIG. 20C may then be applied. As depicted, first pin or rod 326 is further advanced in the direction of Arrow B. However, any further advancement of lever 350 is prevented by flared head 356 of post 342. Assuming lever 340 is constructed of an elastically flexible material, as is preferred, lever finger portion 352 is bent sidewardly, in the direction of Arrow C, about a bend axis 380, so that the finger portion is withdrawn from recess or channel 74d without applying any withdrawal force on secondary IOL edge region 80d. Alternatively, lever finger portion 352 may be withdrawn from recess or channel 74d by lever 340 being axially moved by first pin 326 to the lever retracted position shown in broken lines in FIG. 18.

An additional advantageous feature of instrument 300 is illustrated in FIGS. 21 and 22. This feature enables instrument operating tip assembly 310 to be flexed through an angle, ±α, relative to longitudinal axis 368 of insertion portion 302 between an extreme left-hand position (shown in solid lines in FIG. 21) and an extreme right-hand position (shown in broken lines). The angle, α, is most preferably about 90° (as is depicted in FIG. 21), is preferably at least about 45° (that is, total angular side-to-side movement of tip assembly 310 is preferably at least 90° and is most preferably about 180°). This sidewardly flexing of operating tip assembly 310 enables easy and convenient operation of the operating tip assembly in any quadrant, as described below.

The above-described flexing of operating tip assembly 310 through angle ±α is accomplished by the axial movement of first pin or rod 326 by operating and control system 390 (FIG. 23).

Accordingly, operating tip assembly 310 is flexed toward or to the left hand position (depicted in solid lines in FIG. 21), by axially retracting pin or rod 328 by operating and control system 390 (described below in conjunction with FIG. 23) in the rearward direction of Arrow C′. Conversely, operating tip assembly 310 is flexed toward or to the right hand position (depicted in broken lines in FIG. 21), by axially extending second pin or rod 328 by operating and control system 390 in the forward direction of Arrow C.

Since the axial length of third pin or rod 334 is, as above-described, fixed (i.e., does not change), for second pin or rod 328 to be extended or retracted to cause the flexing of operating tip assembly 310 from one side to the other while still enabling the operation of lever 340 in the manner described above for any position of the tip assembly, the first pin or rod 326 is caused, by operating and control system 390, to be correspondingly extended or retracted as second pin or rod 328 is retracted or extended. This reverse movement of first pin or rod 326 by operating and control system 390 is more particularly described below.

By way of illustrative examples, FIGS. 22A, 22B and 22C depict the use of instrument 300 with operating tip assembly 310 in three different tip assembly positions for inserting differently-located secondary IOL edge regions 80d into a primary IOL annular recess or channel 74d through a common ocular microincision 380 that is not larger than about 2 mm. In FIG. 22A, operating tip assembly 310 is shown in the “straight ahead” position of FIGS. 18 and 19 for inserting a first one of secondary IOL edge regions 80d into primary IOL recess or channel 74d in a first quadrant, ocular insertion portion 302 being shown extending through an ocular microincision 380 not larger than about 2 mm.

For easily inserting a second one of secondary IOL edge regions 80d into primary IOL recess or channel 74d in a third quadrant, operating tip assembly 310 is depicted in FIG. 22B flexed to its extreme left hand position (shown in solid lines in FIG. 21) while ocular insertion portion 302 remains in ocular microincision 380.

For easily inserting a third one of secondary IOL edge regions 80d into primary IOL recess or channel 74d in a second quadrant, operating tip assembly 310 is depicted in FIG. 22B flexed to its extreme right hand position (shown in broken lines in FIG. 21), again while ocular insertion portion 302 remains in ocular microincision 380.

Thus, according to the orientation of secondary IOL 64d relative to primary IOL 62d, and the location and angular extent of secondary IOL edge regions 80d, an operator of instrument 300 can readily flex operating tip assembly 310 to whatever position is best suited for the particular secondary IOL edge region insertion procedure required without having to remove instrument 300 (i.e., ocular incision portion 302) from microincision 380.

With respect to FIG. 22, although primary and secondary IOLs 62d and 64d have been depicted for illustrative purposes, it is to be understood that no limitation to any particular primary or secondary IOLs is thereby intended or implied.

A preferred implementation of operating and control system 390 is depicted in functional form in FIG. 23. First thumb wheel 320 is shown connected to a first pinion gear 400 that engages a first rack gear 402 connected to first pin or rod 326. Thus, turning thumb wheel 320 in a counter clockwise direction causes extension movement of first pin or rod 326 and clockwise turning of the first thumb wheel causes retraction movement of first pin or rod for operation of connected lever 340 as described above in conjunction with FIGS. 18 and 19. For reasons described below, first thumb wheel 320 and first pinion and rack gears 400 and 402 are installed in an inner housing 410 that is, in turn, installed in instrument handle 306.

As shown, second thumb wheel 322 is shown connected to a second pinion gear 412 that engages a second rack gear 414 connected to second pin or rod 328. Thus, turning second thumb wheel 322 in a counter clockwise direction causes extension movement of second pin or rod 328 and clockwise turning of the first thumb wheel causes retraction movement of first pin or rod 326 for causing side-to-side flexing of operating tip assembly 310, as described above relative to FIGS. 21 and 22.

It is further shown in FIG. 23 that second pin or rod 328 is interconnected to inner housing 410 by a sleeve 420 fixed to the second pin or rod, a sleeve 422 fixed to a forward end region of the inner housing and a pivot mechanism 424 connected between the two sleeves.

According to the foregoing, when second pin or rod 328 is retracted (in the direction of Arrow C′ by clockwise rotation of second thumb wheel 322) a certain distance to cause the sideward flexing of tip assembly 310 toward the left hand position depicted in solid lines in FIG. 21, inner housing 410 is caused by pivot mechanism 424 to be moved forwardly a like distance, thereby causing the extension of first pin or rod 326 (direction of Arrow B) with no required rotation of first thumb wheel 320. Conversely, when second pin or rod 328 is extended (in the direction of Arrow C by clockwise rotation of second thumb wheel 322) a certain distance to cause the sideward flexing of tip assembly 310 toward the right hand position depicted in broken lines in FIG. 21, inner housing 410 is caused by pivot mechanism 424 to be moved rearwardly a like distance, thereby causing the retraction of first pin or rod 326 (direction of Arrow B′) with no required rotation of first thumb wheel 320. In either above-described situations, first pin or rod 326 can still be independently (and simultaneously, if desired) extended or retracted by the rotation of first thumb wheel so as to operate lever 340 regardless of the flexed position of tip assembly 310 caused by movement of second pin or rod 328.

Inner housing 410 with first thumb wheel 320, first pinion and rack gears 400 and 402 and first pin or rod 326 may be considered as a first operating and control means 430. Similarly, second thumbwheel 322, second pinion and rack gears 412 and 414, second pin or rod 328, sleeves 420 and 422 and pivot mechanism 424 may be considered as a second operating and control means 432.

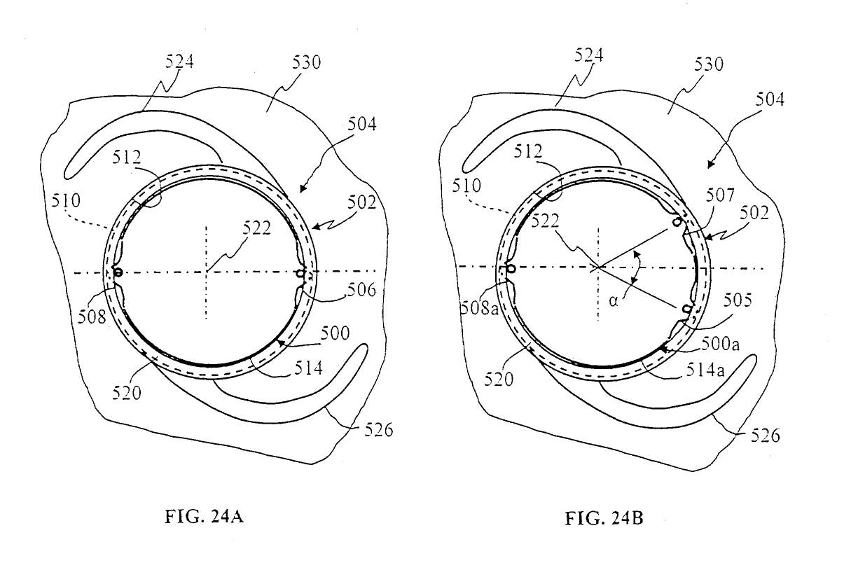

Description of FIGS. 24 Through 32:

In FIG. 24 a secondary optic 500 is shown attached to a primary optic 502 of a primary intraocular lens 504, which may be of either an anterior chamber or posterior chamber type, to modify the optical properties of the primary optic. As shown in FIG. 24A, secondary optic 500 is formed having respective first and second, oppositely located installation or attachment tabs 506 and 508 sized, as more particularly described below, to be installed or inserted into a narrow annular recess 510 formed in preliminary optic 502 relatively adjacent a primary optic peripheral edge 512.

Insertion of secondary optic tabs 506 and 508, which project radially outward from diametrically opposite regions of a secondary optic peripheral edge 514, into primary optic annular recess 510 enables secondary optic 500 to be detachably attached to primary optic 502 to correct or modify the optical properties of the primary optic, as may be needed or desired. To this end, primary optic recess 510, as more particularly described below, is formed generally parallel to an anterior surface 520 of primary optic 502 and opens toward an optical axis 522 of the primary optic. Shown further comprising primary intraocular lens 504 are first and second curved ocular attachment members or haptics 524 and 526, respectively, which position the primary intraocular lens in either the anterior chamber or the posterior chamber of a patient's eye 530.

FIG. 24B is similar in all respects to FIG. 24A, except that a secondary optic 500a is formed having first, second and third installation tabs 505, 507 and 508a. Installation tabs 505 and 507 are shown spaced apart a relatively small distance, defined by an angle, β, which may be about 20 degrees to about 30 degrees, on a peripheral edge region generally opposite to the location of tab 508a.

In any case, all that is required regarding installation tabs 506 and 508 depicted in FIG. 24A and installation tabs 505, 507 and 508a depicted in FIG. 24B is that at least one such installation tab is required for attachment of a secondary optic to primary optic 502.

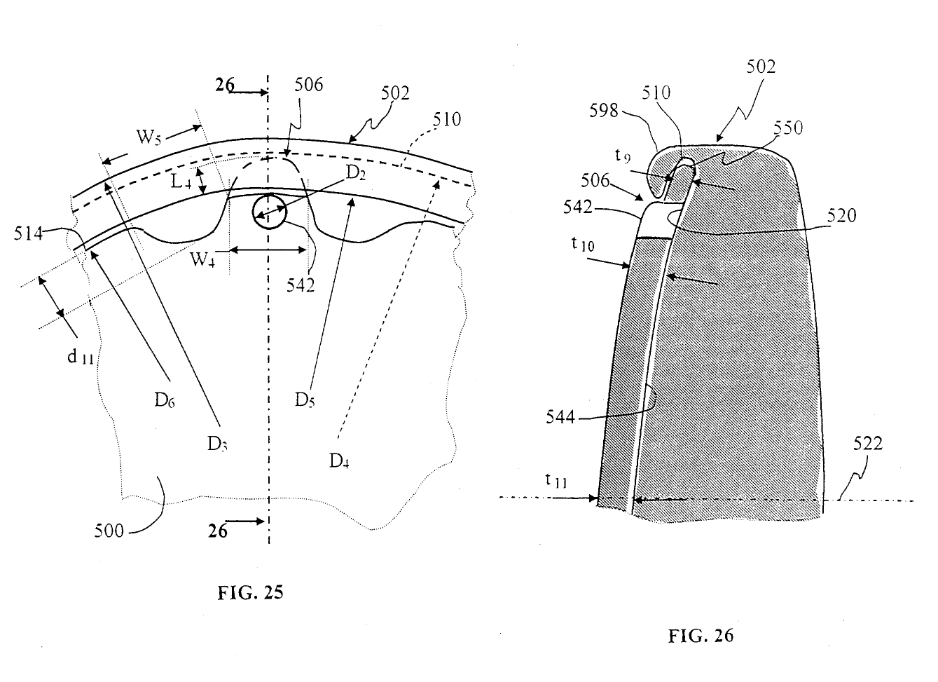

There is shown in FIG. 25 an enlarged plan view of a representative one of the installation tabs, installation tab 506 of secondary optic 500 being depicted by way of example. Formed into peripheral edge 514 of secondary optic 500 at opposite sides of tab 506 are respective first and second arcuate recesses 538 and 540, which, as more particularly described below, facilitate the positioning of secondary optic 500 to insert the tab into primary optic annular recess 510.

Tab 506 is formed having a nominal width, W₄, that is preferably about 0.5 mm and a length, L₄, beyond secondary optic peripheral edge 514 that is preferably about 0.25 mm. Each of recesses 538 and 540 has a width, W₅, that is preferably about 0.4 mm and a depth, d₁₁, below secondary optic peripheral edge 514 that is preferably about 0.2 mm.

Secondary optic tab 506, which is representative of tabs 505, 507 and 508, is formed having a secondary optic manipulation or positioning aperture 542 that has a diameter, D₂ that is preferably about 0.2 mm and which, as depicted in FIG. 25, is located just radially inward of secondary optic periphery edge 514.

It is to be appreciated that dimensions of secondary optic 500 are dependent upon preestablished dimensions of primary optic 502 the optical properties of which are intended to be modified by the secondary optic. For purposes of illustration, with no limitation being intended or implied, let it be assumed that an outer diameter, D₃, of primary optic 502 to peripheral edge 512 is about 6.5 mm and, a diameter, D₄, of the bottom of primary optic annular recess 510, is about 6.3 mm, and a diameter, D₅, of the opening of the annular recess is about 5.8 mm. Corresponding to such exemplary dimensions of primary optic 502, a diameter, D₆, of secondary optic peripheral edge 514 is about 5.6 mm.

As shown in FIG. 26, a posterior surface 544 of secondary optic 500 rests upon primary optic anterior surface 520. An annular recess insertion region 550 of representative secondary optic 500, radially outward of aperture 542, has a thickness, t₉, that is preferably about 0.1 mm, the secondary optic inboard of aperture 552 has a preferred thickness, t₁₀, of about 0.25 mm. A central thickness, t₁₁, of secondary optic 500 at optical axis 522 is preferably not greater than about 0.3 mm.

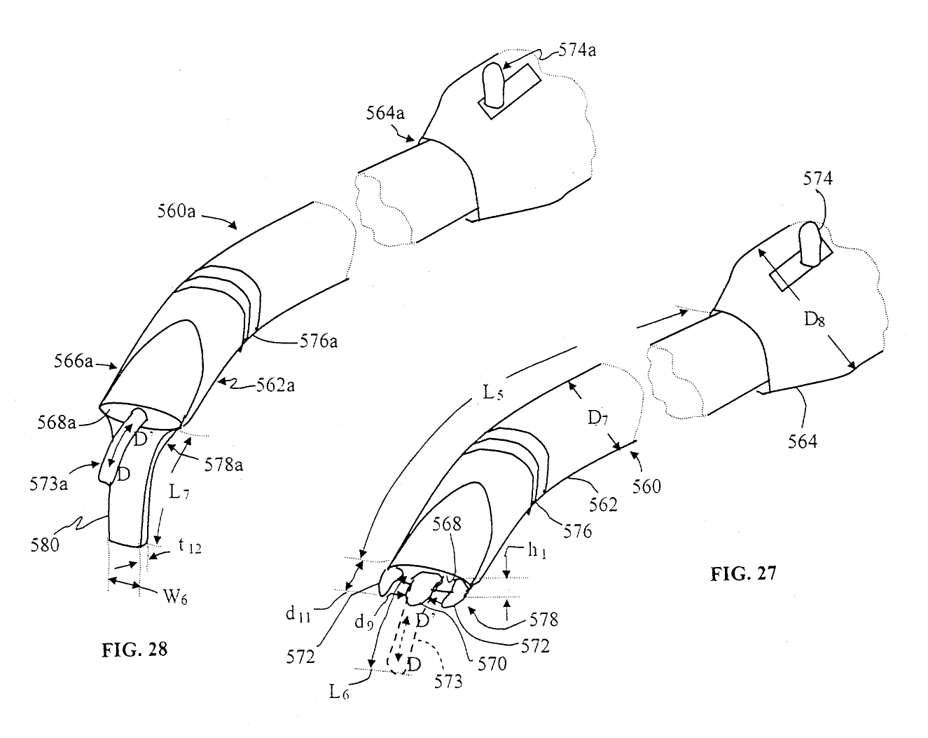

Shown in FIG. 27 is a first secondary optic installation instrument 560 for inserting any selected secondary insertion tab 505, 506, 507, 508 or 508a into primary optic annular recess 510, as more particularly described below.

Comprising first instrument 560 are an elongate slender needle 562 attached to which is a handle 564. Needle 562 has an outside diameter, D₇, that may be about 0.8 mm and a length, L₅, extending from handle 564 that may be about 12.5 mm. Handle 564 has a diameter, D₈, that may be about 7 mm. Preferably, as shown, a distal end region 566 of needle 562 is formed into an oval shape having a height, h₁, of about 0.2 mm. Extending a distance, d₁₁, which is preferably about 0.2 mm, from a distal end 568 of needle is a short, blunt-ended central pin 570 and an opposing pair of small secondary optic guiding or positioning ears or pins 572, located to opposite sides of pin 570. Pin 570 has a diameter, D₉, that is preferably about 0.15 mm, with ears 572 being about the same diameter, but preferably taper to a dull point.

Pin 570 may be short and straight and directly fixed to needle distal end 568 as depicted in solid lines. An alternative pin 570a shown in broken lines, is elongate and is axially slidably installed in needle 562, which is accordingly made hollow for receiving pin 570a. Pin 570a may be extended (direction of arrow “D”) to a length, L₆, which may be about 0.6 mm, from needle distal end 568 by a control 574 in instrument handle 564, and may be retracted (direction of arrow “D′”) to the distance D₁₁, (i.e., about 0.2 mm) so as to be then comparable to pin 570.

Needle 562 is constructed of a biocompatible material, such as stainless steel or titanium, and may be bent as desired at a small angle in a needle region 576. As depicted in FIG. 27, the fully extended portion of elongate pin 573 may be curved, the pin then being constructed of a shape-memory metal material, such as Nitenol, so that when the pin is retracted into needle 562 the pin straightens out and when the pin is subsequently fully extended, the pin reassumes its curved shape.

Pins 570 and 573, ears 572 and needle distal end 568 can, in combination, be considered to comprise a tip 578 of instrument 560.

FIG. 28 depicts a variation secondary optic installation instrument 560a that is preferably identical to above-described secondary optic installation instrument 560 except as specifically described below. As such, variation instrument 560a comprises an elongate hollow slender needle 562a attached to a handle 564a and in which is axially slidably installed an elongate pin 573a, which corresponds directly to above-described elongate pin 573, and which can, thereby, be extended from or retracted into needle 562a. Alternatively, pin 573a can be fixed directly to needle distal end 568a and have a fixed length equal to above-described extended length, L₆, of pin 573 (i.e., about 0.6 mm).

Formed at needle distal end 568a is a slender curved finger 580 that tapers to an end width, W₆, that may be about 0.3 mm, having a thickness, t₁₂, of about 0.15 mm and a having a curved length, L₇, that is preferably about 0.7 mm. Finger 580 is preferably formed as a continuation of needle 562a.

Pin 573a, finger 580 and needle distal end 568a can, in combination, be considered to comprise a tip 578a of instrument 560a.

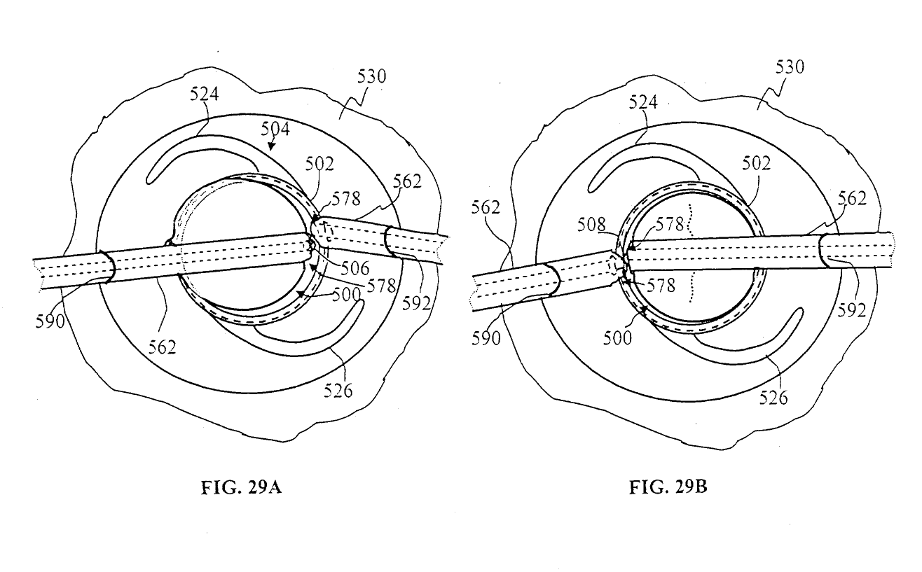

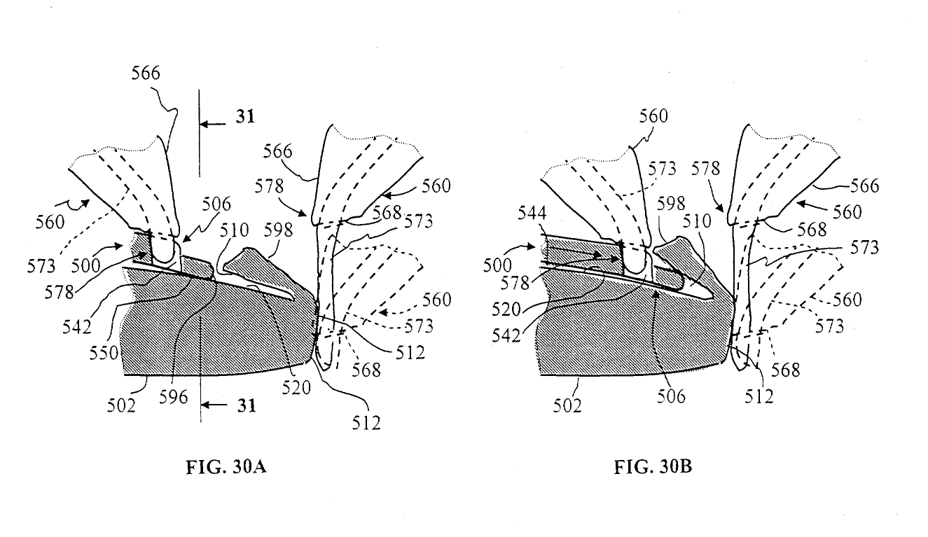

In FIG. 29, there is depicted the manner of inserting tabs 506 and 508 of secondary optic 500 (FIG. 24A) into primary optic annular recess 510. FIG. 29A, which depicts the inserting of secondary optic tab 506 into primary optic annular recess 510, shows first and second small incisions 590 and 592 which are made in eye 530 spaced relatively far apart in horizontal alignment with one another. Secondary optic 500 may be inserted into patient's eye 530 in a rolled condition through one of ocular incisions 590 or 592 and placed onto primary optic anterior surface 520, or may otherwise be inserted into the patient's eye so as to rest on the primary optic anterior surface.

A first installation instrument needle 562 is shown inserted into eye 530 through first incision 590 until positioning aperture 542 in secondary optic tab 506 is engaged by instrument tip 578, as more particularly described below, and secondary optic 500 is positioned by the needle until the tab is positioned adjacent a selected region of primary optic annular recess 510 that is closest to second incision 592.

As depicted, a second one of installation instrument needles 562 (or an instrument needle 562a, not shown) is inserted through second incision 592 and is positioned so that instrument tip 578 (or 578a, as the case may be) bears against primary optic peripheral edge 512 near secondary optic tab 506, to hold primary optic 502 in a fixed position while first instrument needle 562 pushes tab 506 into annular recess 510.

The above procedure is repeated to then insert secondary optic second tab 508 into an opposite region of primary optic annular recess 510 as depicted in FIG. 29A. If both instrument needles 562 are inserted through ocular incisions 590 and 592 to insert secondary optic first tab 506 into primary optic annular recess 510, as described above relative to FIG. 29A, the instrument needles are merely repositioned in their same ocular incisions for inserting second tab 508 into recess 510 in the same manner as the first tab was inserted into the annular recess. Assuming needle 562 was inserted through first incision and if, on the other hand, needle 562a were inserted through incision 592 for installing first tab 506 into primary optic annular recess 510, it would be necessary to shift the two needles so that needle 562 is inserted through second incision 592 and needle 562a is inserted through first incision 590 for installing second tab 508 into the annular recess.

The use of first instrument 560 is disclosed as positioning secondary optic 500 so that tabs 506 and 508 are inserted into primary optic annular recess 510 and second instrument 560 or 560a is disclosed as holding the primary optic in place during the process of inserting the tabs into the annular recess by the first instrument. It is, however, within the scope of the present invention to employ just first instrument 560 to insert tabs 506 and 508 into primary optic annular recess 510 without primary optic being held stationary by second instrument 560 or 560a.

FIG. 30 depicts the use of two identical needles 562 for installing a representative secondary optic tab 506 into primary optic annular recess 510. Elongate pin 573 of one of needles 562 is depicted in its retracted condition in FIG. 30A inserted into secondary optic tab aperture 542 with secondary optic 500 positioned by the needle so the tab is adjacent an entrance 596 to primary optic annular recess 510. Alternatively if needle 562 were provided with short projecting pin 570 (not shown), the short pin would be inserted into secondary optic tab aperture 542 for positioning of secondary optic 500.

Extended pin 573 of the second needle 562 is shown in solid lines in FIG. 30A positioned against primary optic peripheral edge 512 to hold primary optic 502 in place as the first needle 562 pushes the secondary optic tab 506 into annular recess 510 (direction of arrow “E”, FIG. 30B) beneath an overhanging recess lip 598. As an alternative for holding primary optic in place, distal end region 566 of the second needle 562 is shown in broken lines bearing against primary optic edge 512 IN BOTH FIGS. 30A and 30B.

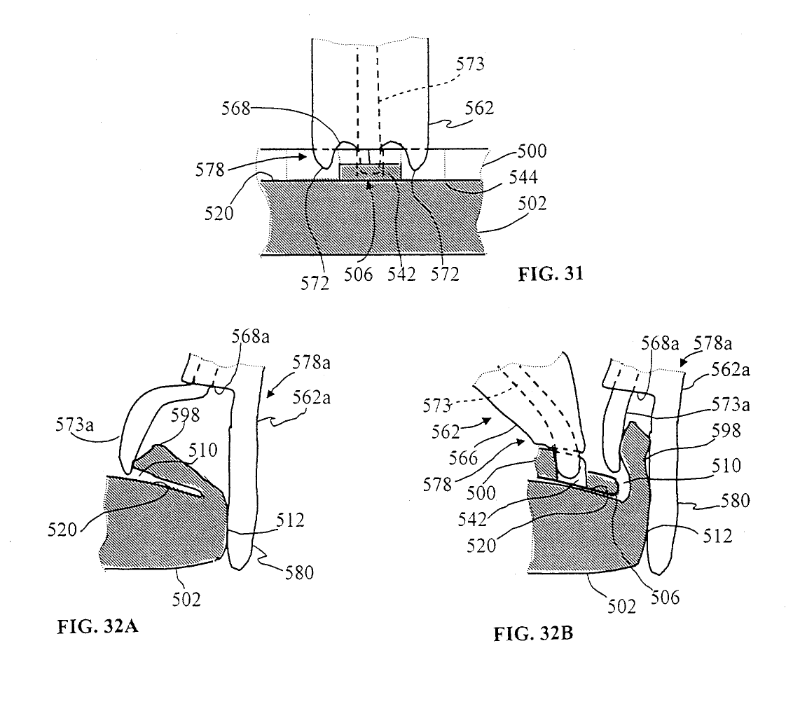

The cross sectional view of FIG. 31 shows the first mentioned needle of FIG. 30 positioned with two ears or pins 572 projecting from opposite side regions of distal end 568 positioned on opposite sides of representative tab 506 of secondary optic 500 with retracted pin 573 inserted into tab aperture 542. Ears or pins 572 importantly assist in moving secondary optic 500 on primary optic anterior surface 520 for proper positioning of secondary optic tab 506 (as well as of tab 508) to and into primary optic annular recess 510.

FIG. 32 shows in two views the insertion of a representative secondary optic tab 506 into a selected region of primary optic annular recess 510 using a needle 562a to open the annular recess foe facilitating the secondary optic tab thereinto. FIG. 32A showing needle 562a positioned with extended pin 703a deflected into lifting engagement with overhanging lip 598 of primary optic annular recess 510 and with finger 580 positioned against primary optic peripheral edge 512 to hold the primary optic in place.

FIG. 32B is a view corresponding generally to FIG. 30B, and showing a representative secondary optic tab 506 positioned by needle 562 for insertion into primary optic annular recess 510, and further showing overhanging lip 598 of recess 510 lifted by needle 562a as depicted in FIG. 32A for ease in tab insertion into the annular recess.

There is accordingly been disclosed a method for attaching a secondary optic 500 to an anterior surface 512 of a primary optic 502 so as to modify the optical characteristics of the primary optic, the primary optic being formed having an annular recess for receiving preferably two installation tabs 506 and 506, and at least one such tab, formed at secondary optic peripheral edge 514.

In summary a preferred general method comprises the steps of:

a. forming a thin, elastically deformable secondary optic 500 having a diameter D₆ substantially equal to a primary optic diameter D₅ at a primary optic recess entrance 596, and having first and second insertion tabs 506 and 508 extending radially outward at generally opposite peripheral edge 514 regions, the tabs being sized for fitting into the primary optic recess;

b. forming a manipulation hole 542 in each of the secondary optic tabs 506 and 508;

c. making small, first and second, spaced far apart ocular incisions 590 and 592 in opposite regions of a patient's eye 530;

d. inserting the secondary optic 500 into patient's eye 530 with a secondary optic posterior surface 544 positioned on a primary optic anterior surface 520, and with first and second tabs 506 and 508 in close proximity with corresponding ones of first and second ocular incisions 590 and 592;

e. forming first and second secondary-optic-to-primary-optic attachment instruments 560 (or 560 and 560a);

f. inserting a first instrument 562 through first incision 590 into patient's eye 30 until a tip 578 thereof engages manipulation hole 542 in first tab 506;

g. positioning secondary optic 500 by first instrument 560 until first tab 506 is adjacent a selected region of primary optic recess 510;

h. inserting second instrument 560 (or 560a) through second ocular incision 592 into patient's eye 530 so that a tip 578a thereof engages primary optic 502 at peripheral edge 512 thereof adjacent first tab 506 for maintaining the primary optic in a fixed position;

i. positioning secondary optic 500 by first instrument 560 so first tab 506 is inserted into primary optic recess 510;

j. repositioning second instrument 560 (or 560a) so that tip 578 thereof engages manipulation hole 542 in second tab 508;

k. repositioning first instrument 560 so that tip 578 thereof engages primary optic peripheral edge 512 adjacent second tab 508 for maintaining the primary optic in a fixed position; and

l. pushing second optic 500 by second instrument 560 (or 560a) until second tab 508 is inserted into primary optic recess 510.

Also disclosed are secondary optic 500 and installation instruments 60 and 560a.

Although there has been described above a secondary optic and instruments for use therewith for modifying n the optical properties of a primary optic of a primary intraocular lens, and an associated method, all in accordance with the present invention, for purposes of illustrating the manner in which the present invention maybe used to advantage, it is to be understood that the invention is not limited thereto. It is thus within the scope of the present invention that a single manufacturer commercially provide the secondary optics, for example, in sets, and in any desired number of styles, configurations, materials and diopter powers, and it is still within the scope of the invention that another manufacturer may provide the installation. Consequently, any and all variations and equivalent arrangements which may occur to those skilled in the applicable art are to be considered to be within the scope and spirit of the invention as set forth in the claims which are appended hereto as part of this application.

What is claimed is:

1. A method for modifying the optical characteristics of a intraocular lens implanted in a patient's eye, the primary intraocular lens having a primary optic with a narrow annular recess formed into an anterior surface generally parallel to said anterior surface and relatively adjacent to a periphery of said primary optic, an entrance to said annular recess having an overhanging lip directed toward an optical axis of said primary optic, said method comprising the steps of:
a. forming a thin, elastically deformable secondary optic having a diameter substantially equal to a primary optic diameter at said primary optic annular recess entrance, and having an insertion tab extending radially outward from a secondary optic peripheral edge, said tab being sized for fitting into said primary optic annular recess;
b. forming a manipulation hole in said tab;
c. making small, first and second, spaced far apart ocular incisions in said patient's eye;
d. inserting said secondary optic into said patient's eye with the secondary optic posterior surface positioned on said primary optic anterior surface, and with said tab in close proximity to one of said first and second ocular incisions;
e. forming a secondary-optic-to- primary-optic attachment instrument;
f. inserting said instrument through said first incision into said patient's eye until a instrument tip thereof engages the manipulation hole in said tab;
g. positioning the secondary optic by said instrument tip until said tab is adjacent a selected region of said primary optic annular recess;
h. positioning the secondary optic by said instrument tip so said tab is inserted into said primary optic annular recess.

2. The method as claimed in claim 1, including the steps of:
a. forming a second secondary optic-to-primary optic attachment instrument; and
b. inserting said second instrument through said second ocular incision into said patient's eye so that an instrument tip thereof engages said primary optic peripheral edge adjacent said tab to maintain the primary optic in a fixed position as the secondary optic is positioned by the tip of the first-mentioned instrument so the tab is inserted into the primary optic annular recess.

3. The method as claimed in claim 2, wherein the step of forming the first-mentioned and second secondary optic-to-primary optic attachment instruments includes forming each of said instruments having an elongate, slender insertion needle and forming a short, axially extending pin and a pair of short axially extending ears to sides of said pin at a distal end of at least said first-mentioned instrument needle.

4. The method as claimed in claim 3, wherein the step of forming the first-mentioned and second secondary optic-to-primary optic attachment instruments includes forming each of said instruments having an elongate, slender insertion needle and forming an elongate, axially extending pin at a distal end of said second instrument needle for engaging and lifting a region of said overhanging lip adjacent said tab to facilitate insertion of the tab into the primary optic recess by the first instrument, and forming an elongate, axially extending tongue at said distal end for engaging the primary optic peripheral edge to hold the primary optic in position as the tab is inserted into the annular recess.

5. The method as claimed in claim 3, wherein the step of forming the first-mentioned and second secondary-optic-to-primary-optic attachment instruments includes forming each of said first and second instruments having an elongate, slender hollow insertion needle and axially slidingly installing a flexible pin in each of said needles, each of said pins being individually extendable and retractable in an associated needle.

6. The method as claimed in claim 5, including forming each of said installed pins with a curved distal end region.

7. The method as claimed in claim 6, including forming each of said installed pins of a shape memory material so that each of said pins can be individually retracted into said associated needle such that a short exposed distal end of each pin is relatively straight and so that when each of said pins is individually extended from said associated needle a longer exposed distal end region of each pin is curved.

8. The method as claimed in claim 1, wherein the step of inserting the secondary optic into said patient's eye includes inserting the secondary optic through one of said first and second ocular incisions.

9. The method as claimed in claim 1, wherein the step of forming a thin, elastically deformable secondary optic, includes forming an arcuate recess in the periphery of said secondary optic adjacent each side of said tab so as to provide a secondary optic guiding region.

10. A method for modifying the optical characteristics of a intraocular lens implanted in a patient's eye, the primary intraocular lens with a primary optic, said primary optic having a narrow annular recess formed into an anterior surface generally parallel to said anterior surface relatively adjacent to a peripheral edge of said primary optic, an entrance to said recess having an overhanging lip directed toward an optical axis of said primary optic, said method comprising the steps of:
a. forming a thin, elastically deformable secondary optic having a diameter substantially equal to a primary optic diameter at said primary optic recess entrance, and having first and second insertion tabs extending radially outward at generally opposite peripheral edge regions, said tabs being sized for fitting into said primary optic recess;
b. forming a manipulation hole in each of said secondary optic first and second tabs;
c. making small, first and second, spaced far apart ocular incisions in opposite regions of said patient's eye;
d. inserting said secondary optic into said patient's eye with the secondary optic posterior surface positioned on said primary optic anterior surface, and with said first and second tabs in close proximity with corresponding ones of said first and second ocular incisions;
e. forming first and second secondary-optic-to-primary-optic attachment instruments;
f. inserting said first instrument through said first incision into said patient's eye until an instrument tip thereof engages the manipulation hole in said first tab;
g. positioning the secondary optic by said first instrument tip until said first tab is adjacent a selected region of said primary optic annular recess;
h. inserting said second instrument through said second ocular incision into said patient's eye so that an instrument tip thereof engages said primary optic peripheral edge adjacent said first tab for maintaining the primary optic in a fixed position; and
i. positioning the secondary optic by the first instrument tip so the first tab is inserted into said primary optic annular recess.

11. The method as claimed in claim 10, wherein the step of forming said first and second instruments includes forming each of the instruments having similar instrument tips and including the further steps of:
a. repositioning said second instrument so that the instrument tip thereof engages the manipulation hole in the second tab;
b. repositioning said first instrument so that the instrument tip thereof engages said primary optic peripheral edge adjacent said second tab for maintaining the primary optic in a fixed position; and
c. pushing said second optic by the second instrument tip until the second tab is inserted into the primary optic annular recess.

12. The method as claimed in claim 10, step of forming said first and second instruments includes forming each of the instruments having different instrument tips and including the further steps of:
a. inserting said first instrument through said second incision into said patient's eye until an instrument tip thereof engages the manipulation hole in said second tab;
b. positioning the secondary optic by said first instrument tip until said second tab is adjacent a selected region of said primary optic annular recess;
c. inserting said second instrument through said first ocular incision into said patient's eye so that an instrument tip thereof engages said primary optic peripheral edge adjacent said second tab for maintaining the primary optic in a fixed position; and
d. positioning the secondary optic by the first instrument tip so the second tab is inserted into said primary optic annular recess.

13. The method as claimed in claim 12, wherein the step of inserting said second instrument through said second and first ocular incisions includes positioning said second instrument so that said instrument tip thereof also engages and lifts a region of said overhanging lip adjacent said first and second tabs so as to facilitate insertion of the first and second tabs into the primary optic recess by the first instrument.

14. The method as claimed in claim 10, wherein the step of forming the first and second secondary optic-to-primary optic attachment instruments includes forming each of said instruments having an elongate, slender insertion needle and forming a short, axially extending pin and a pair of short axially extending ears to sides of said pin at a distal end of at least said first instrument needle.

15. The method as claimed in claim 10, wherein the step of forming said first and second secondary-optic-to-primary-optic attachment instruments includes forming each of said first and second instruments having an elongate, slender hollow needle and axially slidingly installing a flexible pin in each of said needles, each of said pins being individually extendable and retractable in the associated needle and includes forming each of said installed pins with a curved distal end region.

16. The method as claimed in claim 10, wherein the step of forming a thin, elastically deformable secondary optic, includes forming an arcuate recess in the periphery of said secondary optic adjacent each side of each of said first and second tabs so as to facilitate positioning the secondary optic for inserting the first and second tabs into the primary optic annular recess.

17. The method as claimed in claim 10, wherein the step of forming a thin, elastically deformable secondary optic, includes forming the secondary optic from a silicone or acrylic plastic material.

18. A secondary optic for attaching to and modifying the optical characteristics of a intraocular lens implanted in a patient's eye, the primary intraocular lens having a primary optic with a narrow annular recess formed into an anterior surface generally parallel to the anterior surface relatively adjacent to a periphery of said primary optic, an entrance to said annular recess having an overhanging lip directed toward an optical axis of said primary optic, said secondary optic having a diameter substantially equal to a primary optic diameter at said annular recess entrance, said secondary optic formed having at least one insertion tab extending radially outward at a peripheral edge region, said at least one tab being sized for fitting into said primary optic annular recess and having an arcuate recess in the periphery adjacent each side of said at least one tab so as to facilitate positioning said secondary optic for inserting said tab into said annular recess.

19. The secondary optic as claimed in claim 18, including first and second installation tabs extending radially outward at generally opposite peripheral edge regions, said first and second tabs being sized for fitting into said primary optic recess and having an arcuate recess in the periphery adjacent each side of each of said first and second tabs so as to facilitate positioning of said secondary optic for inserting said tabs into said annular recess.

20. The secondary optic as claimed in claim 18, wherein said secondary optic is formed from an elastically deformable material.

21. The secondary optic as claimed in claim 20, wherein said material is selected from the group consisting of silicone and acrylic plastics.

22. The secondary optic as claimed in claim 18, wherein said secondary optic is formed having a secondary optic central thickness no greater than about 0.3 mm.

23. The secondary optic as claimed in claim 19, wherein said first and second tabs each have a width no greater than about 2 mm and extend beyond the secondary optic peripheral edge a distance of no more than about 0.5 mm.

24. The secondary optic as claimed in claim 18, wherein said at least one insertion tab is formed having a small secondary optic positioning aperture.

25. An instrument for installing a peripheral tab of a secondary optic into an annular recess in a primary optic, said secondary optic tab formed having a small positioning aperture, said instrument comprising:
a. an elongate, slender needle having a distal end, said distal end formed having an opposed pair of small, axially-extending, secondary optic holding ears; and
b. a slender pin projecting from said needle distal end between said ears, said pin being sized for insertion into said secondary optic tab positioning aperture.

26. The instrument as claimed in claim 25, wherein said needle is hollow and wherein said pin is axially slidably installed in said needle to enable selective axial extension and retraction of the pin relative to the needle and wherein said pin is formed having an arcuate distal end region.

27. The instrument as claimed in claim 26, wherein said pin is constructed from a shape memory material so as to cause the arcuate distal end region of the pin to straighten when the pin is retracted into the needle and to subsequently restore the arcuate distal end region of the pin when the pin is subsequently extended from the needle.

28. The instrument as claimed in claim 25, wherein said needle is generally cylindrical in shape, wherein said needle distal end having an oval shape and wherein the needle is constructed to enable a distal end region of the needle to be bent at an angle of at least about 10 degrees relative to a remainder of the needle.

29. An instrument for installing a peripheral tab of a secondary optic into an annular recess in a primary optic, said secondary optic tab formed having a small positioning aperture, said instrument comprising:
a. an elongate, slender needle having a distal end, said distal end formed having an elongate slender tongue axially projecting therefrom to enable engagement of a peripheral edge region of said primary optic to prevent movement of the primary optic as the secondary optic tab is installed in said primary optic recess; and
b. a slender pin projecting from said needle distal end, said pin being sized for engaging and lifting an overhanging lip of said primary optic annular recess for facilitating the insertion of said secondary optic tab into said annular recess.

30. The instrument as claimed in claim 29, wherein said needle is generally cylindrical in shape, wherein said needle distal end having an oval shape and wherein the needle is constructed to enable a distal end region of the needle to be bent at an angle of at least about 10 degrees relative to a remainder of the needle.
